(19) United States
(12) Patent Application Publication
(10) Pub. No.: US 2024/0408338 A1
(43) Pub. Date: Dec. 12, 2024

(54) POSITIONING AND STABILISING STRUCTURES FOR PATIENT INTERFACES

(71) Applicant: RESMED ASIA PTE. LTD., Singapore (SG)

(72) Inventors: Thontira SUPAOPASPHUN, Singapore (SG); Bangzheng TAN, Singapore (SG); Mohankumar Krishnan VALIYAMBATH, Singapore (SG); Maximilian Aji WIJOYOSENO, Singapore (SG)

(21) Appl. No.: 18/694,875

(22) PCT Filed: Sep. 23, 2022
(86) PCT No.: PCT/IB2022/058999

§ 371 Date: Mar. 22, 2024

(30) Foreign Application Priority Data
Sep. 24, 2021 (AU) 2021903069

(51) Int. Cl. A61M 16/06

(52) U.S. Cl. CPC A61M 16/0616; A61M 16/0633; A61M 2202/0007; A61M 2202/02; A61M 2205/0216; A61M 2209/088; A61M 2210/0606

(57) ABSTRACT

The invention relates to a positioning and stabilising structure for a patient interface, wherein the positioning and stabilising structure comprises a headgear structure with a pre-defined shape, and a patient interface comprising the positioning and stabilising structure. The invention also relates to methods of manufacturing the headgear structure with a pre-defined shape.

### 1 BACKGROUND OF THE TECHNOLOGY

### 1.1 Field of the Technology

The present technology relates to one or more of the screening, diagnosis, monitoring, treatment, prevention and amelioration of respiratory-related disorders. The present technology also relates to medical devices or apparatus, and their use.

### 1.2 Description of the Related Art

### 1.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD) and Chest wall disorders.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem. See U.S. Pat. No. 4,944,310 (Sullivan).

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterised by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload. See U.S. Pat. No. 6,532,959 (Berthon-Jones).

Respiratory failure is an umbrella term for respiratory disorders in which the lungs are unable to inspire sufficient oxygen or exhale sufficient CO2 to meet the patient's needs. Respiratory failure may encompass some or all of the following disorders.

A patient with respiratory insufficiency (a form of respiratory failure) may experience abnormal shortness of breath on exercise.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

A range of therapies have been used to treat or ameliorate such conditions. Furthermore, otherwise healthy individuals may take advantage of such therapies to prevent respiratory disorders from arising. However, these have a number of shortcomings.

### 1.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 1.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube or endotracheal tube. In some forms, the comfort and effectiveness of these therapies may be improved.

### 1.2.2.2 Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that may be held approximately constant throughout the respiratory cycle. The treatment flow rate is nominally set to exceed the patient's peak inspiratory flow rate. HFT has been used to treat OSA, CSR, respiratory failure, COPD, and other respiratory disorders. Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

### 1.2.2.3 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

Certain other mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth.

Certain masks may be uncomfortable or impractical for the present technology if they require a patient to insert a portion of a mask structure in their mouth to create and maintain a seal via their lips.

Certain masks may be impractical for use while sleeping, e.g. for sleeping while lying on one's side in bed with a head on a pillow.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

As a consequence of these challenges, some masks suffer from being one or more of obtrusive, aesthetically undesirable, costly, poorly fitting, difficult to use, and uncomfortable especially when worn for long periods of time or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. This discomfort may lead to a reduction in patient compliance with therapy. This is even more so if the mask is to be worn during sleep.

CPAP therapy is highly effective to treat certain respiratory disorders, provided patients comply with therapy. If a mask is uncomfortable, or difficult to use a patient may not comply with therapy. Since it is often recommended that a patient regularly wash their mask, if a mask is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

### 1.2.2.3.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

Another form of seal-forming structure may use adhesive to achieve a seal. Some patients may find it inconvenient to constantly apply and remove an adhesive to their face.

A range of patient interface seal-forming structure technologies are disclosed in the following patent applications, assigned to ResMed Limited: WO 1998/004,310; WO 2006/074,513; WO 2010/135,785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Limited has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications, assigned to ResMed Limited, describe examples of nasal pillows masks: International Patent Application WO2004/073,778 (describing amongst other things aspects of the ResMed Limited SWIFT™ nasal pillows), US Patent Application 2009/0044808 (describing amongst other things aspects of the ResMed Limited SWIFT™ LT nasal pillows); International Patent Applications WO 2005/063,328 and WO 2006/130,903 (describing amongst other things aspects of the ResMed Limited MIRAGE LIBERTY™ full-face mask); International Patent Application WO 2009/052,560 (describing amongst other things aspects of the ResMed Limited SWIFT™ FX nasal pillows).

### 1.2.2.3.2 Positioning and Stabilising

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face.

One technique is the use of adhesives. See for example US Patent Application Publication No. US 2010/0000534. However, the use of adhesives may be uncomfortable for some.

Another technique is the use of one or more straps and/or stabilising harnesses. Many such harnesses suffer from being one or more of ill-fitting, bulky, uncomfortable and awkward to use.

Headgear or headgear arrangements may be used to position and stabilise the seal-forming structure of the patient interface on the patient's face. The headgear is generally flexible and can be bent so that it fits and conforms to the shape of the patient's head and/or face when using the patient interface. However, when the headgear is bent is can create creases or wrinkles, which may be uncomfortable and affect the fit of the patient interface. This may reduce patient compliance with therapy. Flexible headgear does not hold its shape well. It can become tangled and is difficult to use. Again, this discourages patient compliance.

Some existing headgear is formed with a predefined shape. A flat component may be shaped to a predefined. 3D shape. In other words, the flat component may be shaped so that it extends in directions along different planes. But in doing so, creases or wrinkles are created. Some existing headgear may be rigidised or supported to hold the predefined shape. For instance, the headgear may include support structures or rigidisers provided to a headgear material or materials e.g. textiles or fabrics. The support structures or rigidisers have a predefined shape or are formed into a shape so that headgear material adopts the shape of the support structure or rigidiser. The headgear material does not have a predefined shape. Therefore, the support structure or rigidiser may help the headgear material hold its shape. However, the headgear material can crease or wrinkle as it is bent by the rigidiser or support structure.

Furthermore, it may be difficult, time consuming and/or expensive to manufacture headgear with a predefined shape and that does not crease or wrinkle when bent.

Some existing headgear may have edges, seams and/or joints on them. For instance, seams or joints which attach different parts of the headgear structure together, e.g. a first half of the headgear structure which forms a patient facing or contacting side, in use, and a second half of the headgear structure which forms a non-patient facing side. These edges, seams or joints can be rough, uneven, sharp and/or obtrusive. They may irritate and mark the patient's skin which reduce compliance with therapy. This may occur when the edges, seams and/or joints are located or positioned, in use, such that they contact the patient's skin and/or head. In addition, these edges, seams or joints may also be unsightly and reduce the aesthetics of the product which could further reduce compliance.

### 1.2.2.4 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

The designer of a device may be presented with an infinite number of choices to make. Design criteria often conflict, meaning that certain design choices are far from routine or inevitable. Furthermore, the comfort and efficacy of certain aspects may be highly sensitive to small, subtle changes in one or more parameters.

### 1.2.2.5 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 1.2.2.6 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

### 1.2.2.7 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

### 2 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

In another aspect of the present technology there is provided a positioning and stabilising structure configured to hold a seal-forming structure of a patient interface in a sealing position on a patient's face. The positioning and stabilising structure may comprise a headgear structure. The headgear structure may be rigidised. The headgear structure may be configured with a predefined shape.

In another aspect of the present technology there is provided a positioning and stabilising structure configured to hold a seal-forming structure of a patient interface in a sealing position on a patient's face. The positioning and stabilising structure may comprise a headgear structure. The headgear structure may comprise a sleeve. The headgear structure may comprise a thermoformable layer which is thermoformed to the sleeve.

In examples:
The headgear structure may be a hollow headgear structure.
The headgear structure may be configured to extend, in use, over a portion of the patient's face and/or head.
The sleeve may comprise an outer surface and an inner surface.
The outer surface of the sleeve may form an outer surface of the headgear structure which contacts the patient's face and/or head in use.
The outer surface of the sleeve may be continuous.

In examples:
The thermoformable layer may be thermoformed to the inner surface of the sleeve.
The thermoformable layer may be thermoformed to the inner surface of the sleeve to provide a pre-defined shape to the sleeve.
The headgear structure may comprise an inner surface which surrounds a cavity extending through at least a portion of the length of the headgear structure.
The thermoformable layer may be thermoformed to the inner surface of the sleeve to form an inner surface of the headgear structure which surrounds a/the cavity extending through at least a portion of the length of the headgear structure.

In examples:
The outer surface of the sleeve may be configured to be smooth, seamless and/or joint free.
The inner surface of the sleeve may be continuous.
The inner surface of the sleeve may be configured to be smooth, seamless and/or joint free.
The sleeve may be a continuous structure, when viewed in cross-section from a side in a direction substantially parallel to a longitudinal axis of the headgear structure.
The headgear structure may comprise a headgear strap or tie.
The thermoformable layer may comprise a thermoformable material.
The thermoformable material may comprise a thermo-fusible yarn.
The thermoformable layer may be positioned on the inner surface of the sleeve such that the thermoformable layer covers at least a portion of the inner surface of the sleeve.
The thermoformable layer may be positioned on the inner surface of the sleeve such that the thermoformable layer covers a substantial portion of the sleeve.
The thermoformable layer may be positioned on the inner surface of the sleeve such that the thermoformable layer covers the entire inner surface of the sleeve to form an inner surface of the headgear structure.
The thermoformable layer may be a continuous structure, when viewed in cross-section from a side in a direction substantially parallel to a longitudinal axis of the headgear structure.
The headgear structure may comprise a patient facing side and a non-patient facing side, wherein at least one of the patient facing side and the non-patient facing side is substantially convex shaped.
The thermoformable layer may have a lower melting point than a layer of the sleeve which provides the inner surface of the sleeve.
The thermoformable layer, when thermoformed to the inner surface of the sleeve, may configure the headgear structure to be substantially resilient or to act resiliently such that the headgear structure returns to the predefined shape when flexed, bent, and/or compressed from the predefined shape.
The headgear structure may be configured to stretch in a first direction and restrict stretching in a second direction, wherein the first direction is different to the second direction.
The sleeve may be configured to stretch in directions which extend along the length and width of the headgear structure.
The thermoformable layer may be configured to stretch in a direction or directions which extend along the length of the headgear structure and restrict stretching in a direction or directions which extend along the width of the headgear structure.
The thermoformable material, e.g. the thermo-fusible yarn, may be positioned on the thermoformable layer to extend along at least a portion of the width of the thermoformable layer to restrict stretching along the width of the headgear structure.

In examples:
The headgear structure may further comprise a rigidiser component.
The rigidiser component may be positioned inside a/the cavity formed in the headgear structure.
The rigidiser component and the headgear structure may be permanently attached or formed together.
The rigidiser component and the headgear structure may be removably attached to each other.

In examples:
A first region of the headgear structure may be configured to be harder, stiffer or more rigid than a second region of the headgear structure.
A section of the thermoformable layer located in the first region of the headgear structure may comprise a greater amount of a/the thermoformable material with respect to a section of the thermoformable layer located in the second region of the headgear structure.

In another aspect of the present technology there is provided a positioning and stabilising structure configured to hold a seal-forming structure of a patient interface in a sealing position on a patient's face. The positioning and stabilising structure may comprise a headgear structure. The headgear structure may comprise a sleeve. The headgear structure may comprise a core positioned inside the sleeve.

In examples:
The sleeve may comprise an outer surface and an inner surface.
The outer surface of the sleeve may form an outer surface of the headgear structure which contacts the patient's face and/or head in use.
The sleeve may comprise an outer layer.
The outer layer may be configured to provide the outer surface of the sleeve.
The sleeve may comprise an inner layer.
The inner layer may be configured to provide the inner surface of the sleeve.
The inner surface of the sleeve may form an inner surface of headgear structure.
The inner layer may be positioned radially inwards from the outer layer.
The inner layer may be less permeable than the outer layer.

In examples:
The core may be positioned inside the sleeve to provide a pre-defined shape to the sleeve.
The core may be positioned inside the sleeve between portions of the inner layer to provide a pre-defined shape to the sleeve.

In examples:
The inner layer may be substantially impermeable.
The core is formed inside the sleeve from a soft material positioned inside the sleeve which hardens to form the core.
The inner layer may limit or prevent the soft material from flowing into the outer layer before it hardens to form the core.
The inner layer and the outer layer may be attached together.
The headgear structure may comprise a patient facing side and a non-patient facing side.
At least one of the patient facing side and the non-patient facing side of the headgear structure may be substantially convex-shaped, when viewed in cross-section from a side in a direction substantially parallel to a longitudinal axis of the headgear structure.
The sleeve may comprise a first portion and a second portion.
Each first portion and second portion may comprise lateral end regions.
The lateral end regions may extend along the length of the respective first portion and the second portion.
The first portion and the second portion may be attached to each other at the respective lateral end regions.
The first portion may be configured to provide a/the patient facing side of the headgear structure.
The second portion may be configured to provide a/the non-patient facing side of the headgear structure.
The respective lateral end regions of the first portion and the second portion may be attached to each other using an adhesive.
The second layer may limit or prevent the adhesive from flowing into the outer layer before the adhesive sets, hardens or cures.

Another aspect of the present technology relates to a positioning and stabilising structure configured to hold a seal-forming structure of a patient interface in a sealing position on a patient's face. The positioning and stabilising structure may comprise a headgear structure. The headgear structure may be configured to extend, in use, over a portion of the patient's face and/or head. The headgear structure may comprise a sleeve. The sleeve may comprise an outer surface and an inner surface. The outer surface of the sleeve may form an outer surface of the headgear structure which contacts the patient's face and/or head in use. The headgear structure may comprise a thermoformable layer. The thermoformable layer may be thermoformed to the inner surface of the sleeve. The thermoformable layer may be thermoformed to the inner surface of the sleeve to provide a pre-defined shape to the sleeve. The thermoformable layer may be thermoformed to the inner surface of the sleeve to form an inner surface of the headgear structure which surrounds a cavity extending through at least a portion of the length of the headgear structure. The outer surface of the sleeve may be continuous.

Another aspect of the present technology relates to a positioning and stabilising structure. The positioning and stabilising structure may be configured to hold a seal-forming structure of a patient interface in a sealing position on a patient's face. The positioning and stabilising structure may comprise a headgear structure. The headgear structure may be configured to extend, in use, over a portion of the patient's face and/or head. The headgear structure may comprise a sleeve. The sleeve may comprise an outer surface and an inner surface. The outer surface of the sleeve may form an outer surface of the headgear structure which contacts the patient's face and/or head in use. The sleeve may comprise an outer layer. The outer layer may be configured to provide the outer surface of the sleeve. The sleeve may comprise an inner layer. The inner layer may be configured to provide the inner surface of the sleeve. The inner surface of the sleeve may form an inner surface of headgear structure. The inner layer may be positioned radially inwards from the outer layer. The inner layer may be less permeable than the outer layer. The headgear structure may further comprise a core. The core may be positioned inside the sleeve to provide a pre-defined shape to the sleeve. The core may be positioned inside the sleeve between portions of the inner layer to provide a pre-defined shape to the sleeve.

Another aspect of the present technology relates to a patient interface. The patient interface may comprise a plenum chamber. The plenum chamber may be pressurisable to a therapeutic pressure of at least 6 cmH2O above ambient air pressure. The plenum chamber may comprise an inlet. The inlet may be configured to receive a flow of air at the therapeutic pressure for breathing by a patient. The patient interface may comprise a seal-forming structure. The seal-forming structure may be configured to form a seal with a region of the patient's face surrounding an entrance to the patient's airways. The seal-forming structure may be configured to maintain a/said therapeutic pressure in the plenum chamber throughout the patient's respiratory cycle in use. The patient interface may comprise a positioning and stabilising structure according to any one or more of the previously described aspects and/or examples of the invention.

According to an aspect of the present technology there is provided a positioning and stabilising structure comprising a headgear structure formed in a predefined shape.

According to another aspect of the present technology there is provided a patient interface, wherein the patient interface comprises:
a plenum chamber pressurisable to a therapeutic pressure of at least 6 cmH₂O above ambient air pressure, wherein the plenum chamber comprises an inlet configured to receive a flow of air at the therapeutic pressure for breathing by a patient;
a seal-forming structure configured to form a seal with a region of the patient's face surrounding an entrance to the patient's airways, wherein the seal-forming structure is configured to maintain said therapeutic pressure in the plenum chamber throughout the patient's respiratory cycle in use; and
a positioning and stabilising structure comprising a headgear structure formed in a predefined shape.

In an example, the headgear structure may comprise a sleeve.

In an example, the sleeve may be a continuous sleeve.

In an example, the headgear structure may comprise at least one layer. For instance, the sleeve may be formed from at least one layer of material.

In an example, the headgear structure may comprise a rigidiser,

In an example, the rigidiser may be thermoformed to at least one of the layers that form the sleeve to provide a pre-defined shape to the sleeve.

In an example, the headgear structure may comprise at least one thermoformable layer. For instance, the thermoformable layer may provide the rigidiser.

In an example, the headgear structure may comprise the thermoformable layer and at least one other layer.

In an example, the thermoformable layer may be thermoformed to at least one other layer to provide the pre-defined shape to the headgear structure.

In an example, the headgear structure may comprise a core inside the sleeve.

In an example, the sleeve may comprise a first layer and a second layer.

In an example, the second layer may be arranged to define a cavity in the sleeve.

In an example, the first layer may be less permeable than the second layer e.g. less permeable to a liquid material.

In an example, the core may at least be partially integrated into a portion of the sleeve which defines the cavity.

According to another aspect of the present technology there is provided a positioning and stabilising structure for a patient interface, wherein the positioning and stabilising structure comprises:
a headgear structure comprising:
   a sleeve formed from at least one layer of material, and
   a rigidiser,
wherein the rigidiser is thermoformed to at least one of the layers that form the sleeve to provide a pre-defined shape to the sleeve.

According to another aspect of the present technology there is provided a patient interface, wherein the patient interface comprises:
a plenum chamber pressurisable to a therapeutic pressure of at least 6 cmH₂O above ambient air pressure, wherein the plenum chamber comprises an inlet configured to receive a flow of air at the therapeutic pressure for breathing by a patient;
a seal-forming structure configured to form a seal with a region of the patient's face surrounding an entrance to the patient's airways, wherein the seal-forming structure is configured to maintain said therapeutic pressure in the plenum chamber throughout the patient's respiratory cycle in use; and
a positioning and stabilising structure which comprises a headgear structure, wherein the headgear structure comprises:
   a sleeve formed from at least one layer of material, and
   a rigidiser,
wherein the rigidiser is thermoformed to at least one of the layers that form the sleeve to provide a pre-defined shape to the sleeve.

Throughout the present specification, reference to the term “continuous” should be understood to mean that the headgear structure and one or more of its components, e.g. the sleeve is smooth, and/or does not have a seam or joint e.g. which extends along its length, or part thereof. For instance, the sleeve may be formed using a circular knitting or other technique to form a closed loop structure or a continuous structure. Therefore, a perimeter or circumference of one or more surfaces, e.g. an inner and/or outer surface, of the headgear structure, or components thereof, which extend along at least a portion of the length thereof, are continuous. That is, the perimeter or circumference is smooth, seamless and/or joint-free. Reference to “smooth” throughout the specification may be understood to refer to a surface that is substantially even, meaning the surface is free from projections or protrusions and/or notches, grooves, recesses, or indentations, or other interruptions on the surface, and which may create sharp, rough, or uneven surfaces, edges, joints, and/or seams on the surface. This should become clearer from the following description.

In an example, at least one of the layers that form the sleeve may comprise a first layer.

In an example, the rigidiser may comprise a thermoformable layer.

In an example, the sleeve may comprise the thermoformable layer.

In an example, the thermoformable layer may be positioned with respect to the first layer to extend over at least a portion of an inner surface of the first layer. In a preferred example, the first layer extends over substantially the entire inner surface of the of the first layer.

In an example, the thermoformable layer may form an inner layer and the first layer may form an outer layer which contacts, in use, the patient's skin and/or hair.

In an example, the first layer may be soft. For instance, the first layer may comprise a soft material.

In an example, the first layer may be breathable. For instance, the first layer may comprise a breathable material.

In an example, the first layer may stretch in one or more directions. For instance, the first layer may be constructed from one or more elastic materials e.g. an elastic yarn.

In an example, the first layer may comprise a polyamide material. For instance, the first layer may comprise nylon.

In an example, the first layer may comprise a polyurethane material. For instance, the first layer may comprise a polyurethane co-polymer material such as elastane (e.g. spandex, Lycra®, or ROICA™), amongst others.

In an example, the thermoformable layer may comprise a thermoformable material. For instance, the thermoformable material may comprise at least one of polypropylene, polyethylene, thermoplastic polyurethane, and a thermoplastic elastomer.

In an example, the thermoformable layer may stretch in one or more directions. For instance, the thermoformable layer may be constructed from one or more elastic materials e.g. an elastic yarn.

In an example, the thermoformable material comprises a thermo-fusible yarn.

In an example, the thermoformable layer may comprise at least one support element that supports the thermoformable material. For instance, the support element may comprise a support yarn that supports the thermo-fusible yarn.

In an example, the support element may comprise at least one of nylon and polyester.

In an example, the thermoformable material may have a lower softening temperature than the support element. For instance, the thermo-fusible yarn may have a lower melting point than the support yarn.

In an example, the sleeve may comprise a double knit structure. For instance, the headgear structure may comprise a double knit fabric.

In an example, the double knit structure may comprise a technical face and a technical back, wherein the technical face may be formed by a first knit layer, and the technical back may be formed by a second knit layer, and wherein the first knit layer and the second knit layer may be knitted together. For instance, the first knit layer may provide the first layer and the second knit layer may provide the thermoformable layer.

In an example, the thermo-fusible yarn may be knitted or woven with the support yarn. For instance, one of the warp thread and the weft thread may comprise the support yarn, and the other one of the warp thread and the weft thread may comprise a thermo-fusible yarn.

In an example, the thermo-fusible yarn may be covered or twisted with the support yarn.

The density and/or thickness of at least one of the thermo-fusible yarn and the support yarn may be selected to provide a stiffer, more rigid headgear structure, or to provide a more flexible, softer headgear structure.

In an example, the thermo-fusible yarn may be warped with the support yarn.

In an example, the thermo-fusible yarn may be braided with the support yarn.

In an example, the headgear structure can be positioned on a shaping component with a predefined shape, e.g. a mould or a mandrel with a predefined shape.

In an example, the rigidiser thermoformable layer can be heated to a temperature at which it will deform. For instance, the thermoformable layer can be heated to a temperature at which the thermoformable material will deform.

In an example, the rigidiser can be heated by the shaping component to a temperature at which the rigidiser will deform to take the shape of the shaping component. For instance, the thermoformable layer can be heated by a mandrel to a temperature at which the thermoformable material will deform to take the shape of the mandrel.

In an example, the headgear structure may be configured to limit or prevent the creation of creases or wrinkles on the headgear structure when forming the headgear structure or sleeve into the predefined shape.

In an example, the headgear structure may stretch in one or more directions. For instance, the sleeve may comprise a stretchable first layer and thermoformable layer as described herein.

In an example, the headgear structure may stretch from a neutral configuration to a stretched configuration when positioning the headgear structure on the shaping component, e.g. the mandrel.

In an example, the headgear structure positioned on the shaping component in the stretched configuration may be heated to a/the temperature at which the rigidiser will deform to maintain the headgear structure in the stretched configuration. For instance, the headgear structure positioned on the mandrel in the stretched configuration may be heated to a/the temperature at which the thermoformable material will deform to maintain the headgear structure in the stretched configuration.

In an example, the headgear structure may flex, bend, and/or be compressed from the predefined shape.

In an example, the headgear structure may be substantially flexible.

In an example, the headgear structure may be configured to return to the predefined shape when flexed, bent, and/or compressed from the predefined shape. For instance, the headgear structure may be substantially resilient or configured to act resiliently.

In an example, the headgear structure may stretch in at least a first direction, and restrict stretching in at least a second direction.

In an example, the thermoformable layer may stretch in at least a first direction, and restrict stretching in at least a second direction. For instance, the thermoformable layer may stretch along its length and restrict stretching along its width.

In an example, the thermoplastic material, e.g. the thermoplastic element, may extend across at least a portion of the width of the thermoformable layer. For instance, the thermo-fusible yarn may extend across the entire width of the thermoformable layer.

In an example, the first layer may stretch in at least a first direction, and restrict stretching in at least a second direction. For instance, the first layer may stretch along its length and restricts stretching along its width.

In an example, the headgear structure may be configured to limit or prevent the formation of sharp edges, seams or joints. For instance, the headgear structure may comprise a continuous sleeve.

In an example, the sleeve may be a continuous sleeve.

In an example, the headgear structure may comprise a substantially oval-shaped cross-section when viewed in a plane substantially parallel to its length.

In an example, the headgear structure may comprise a non-oval shaped cross-section when viewed in a plane substantially parallel to its length. For instance, the cross-section may be any other suitable shape that does not create sharp edges or joints. The cross-section shape may be obround, circular, or polygonal with rounded corners or edges.

In an example, the first layer may be formed in a continuous sleeve.

In an example, the thermoformable layer may formed in a continuous sleeve.

In an example, the thermoformable layer and the first layer may be attached or formed together.

In an example, the thermoformable layer and the first layer may be attached or formed together before the thermoformable layer is heated. For instance, the thermoformable layer and the first layer may be integrally formed, e.g. in the continuous sleeve.

In an example, the thermoformable layer the first layer may be thermoformed together when heated to a/the temperature at which the thermoformable material will deform.

In an example, the thermoformable layer may have a lower melting point than the first layer.

In an example, the headgear structure may be substantially lightweight compared to existing headgear structures with predefined shapes. For instance, the continuous sleeve may create a lighter headgear structure.

In an example, the headgear structure may be configured to limit or prevent flexing, bending, and/or compression in one or more regions. For instance, the headgear structure may be relatively rigid or rigidised in one or more regions.

In an example, the headgear structure may be relatively rigid or rigidised in one or more regions by the thermoformable layer when thermoformed to the at least one other layer of the headgear structure.

In an example, at least one section of the headgear structure may be relatively rigid or rigidised with respect to at least one other section of the headgear structure.

In an example, a section of the thermoformable layer located in the at least one section of the headgear structure may comprise a greater amount of thermoformable material with respect to another section of the thermoformable layer located in the at least one other section of the headgear structure.

In an example, the headgear structure may comprise at least one rigidiser component.

In an example, the headgear structure may be configured to hold at least one rigidiser component. For instance, the continuous sleeve may define a cavity in which the rigidiser component can be positioned.

In an example, the rigidiser component may have a predefined shape.

In an example, the rigidiser component may contour or otherwise correspond to the predefined shape of the headgear structure in at least one plane.

In an example, at least a portion of the rigidiser component may be relatively flat or planar and at least a portion may be curved.

In an example, the rigidiser component may be relatively thin.

In an example, the rigidiser component and the sleeve may be attached or formed together.

In an example, the rigidiser component(s) may be provided to the at least one section of the headgear structure.

According to another aspect of the present technology there is provided a positioning and stabilising structure for a patient interface, the positioning and stabilising structure comprises:
a headgear structure which comprises:
   a sleeve, and
   a core inside the sleeve,
wherein the sleeve comprises a first layer and a second layer,
wherein the second layer is arranged to define a cavity in the sleeve,
and wherein the second layer is less permeable than the first layer, and
wherein the core is at least partially integrated into a portion of the second layer which defines the cavity.

According to another aspect of the present technology there is provided a patient interface, wherein the patient interface comprises:
a plenum chamber pressurisable to a therapeutic pressure of at least 6 cmH₂O above ambient air pressure, wherein the plenum chamber comprises an inlet configured to receive a flow of air at the therapeutic pressure for breathing by a patient;
a seal-forming structure configured to form a seal with a region of the patient's face surrounding an entrance to the patient's airways, wherein the seal-forming structure is configured to maintain said therapeutic pressure in the plenum chamber throughout the patient's respiratory cycle in use; and
a positioning and stabilising structure comprising a headgear structure which comprises:
   a sleeve, and
   a core inside the sleeve,
wherein the sleeve comprises a first layer and a second layer,
wherein the second layer is arranged to define a cavity in the sleeve,
and wherein the second layer is less permeable than the first layer, and
wherein the core is at least partially integrated into a portion of the second layer which defines the cavity.

In an example, the first layer may be similar to the first layer described herein.

In an example, the second layer may be positioned with respect to the first layer to extend over at least a portion of an inner surface of the first layer. In a preferred example, the second layer extends over the entire inner surface of the first layer.

In an example, the second layer may form an inner layer and the first layer may form an outer layer which contacts the patient's skin and/or hair in use.

In an example, the second layer may be configured to limit or prevent the core material and/or adhesives from flowing into the first layer.

In an example, the second layer may be substantially impermeable. For instance, the second layer may comprise a gas and/or liquid impermeable material.

In an example, the headgear structure may stretch in one or more directions.

In an example, the first layer may stretch in one or more directions. For instance, the first layer may be substantially elastic as described herein.

In an example, the second layer may stretch in one or more directions. For instance, the second layer may be substantially elastic. The second layer may comprise an elastic material.

In an example, the second layer and the first layer may be attached or formed together. For instance, the second layer and the first layer may be at least one of welded, laminated, thermo-bonded, stitched or adhered together.

In an example, the core may comprise a core material. For instance, the core material includes a soft material which can harden.

In an example, the core material may be heat treated to melt the core material, which can then cool to harden. For instance, the core material may be a powder, e.g. a resin powder.

In an example, the core material may be a soft solid material, a liquid or a foam which can cure or set to harden, e.g. a liquid polymer, or a polyurethane foam.

In an example, the soft solid material, the liquid or the foam may be thermoset.

In an example, the core material may be positioned in the cavity. For instance, the core material may be injected into the cavity.

In an example, the core material may flow into the second layer and cure. For instance, the core material may be melt and integrate into the fibres forming the second layer, and subsequently harden on curing to form a bond between the rigidiser and the material.

In an example, the hardened core may provide a desired shape to the headgear structure.

In an example, the headgear structure may be shaped using a shaping component. For instance, the sleeve and the core material positioned in the cavity may be moulded into the predefined shape using a mould with a predefined shape.

In an example, the headgear structure may be shaped to position in use any sharp edges or joints away from the patient's head and/or face contacting portion. For instance, the headgear structure may comprise a substantially oval-shaped cross-section when viewed in a plane substantially parallel to its length, or a substantially lens-shaped cross-section when viewed in a plane substantially parallel to its length.

In an example, the headgear structure may comprise a first portion and a second portion which are joined to form the sleeve. For instance, the first portion and the second portion may each comprise edge or end regions which may be attached or formed together to form the sleeve.

In an example, the first portion and the second portion each comprise the first layer and the second layer.

According to another aspect of the present technology there is provided a method of manufacturing a headgear structure as described herein, the method comprising the following steps, occurring in any order:
a) forming a headgear structure; and
b) shaping the headgear structure.

In examples, step (a) comprises:
forming the first layer as described herein. For instance, this step may include weaving or knitting the first layer.
forming the thermoformable layer as described herein. For instance, this step may include weaving or knitting the thermoformable layer from a thermo-fusible yarn and a support yarn.
positioning the thermoformable layer against the first layer. For instance, this step may include forming or attaching the thermoformable layer and the first layer together.
arranging the second layer to form the cavity for the sleeve.
forming the sleeve comprising the first layer and the second layer as described herein.
positioning the second layer against the first layer. For instance, this step may include forming or attaching the second layer and the first layer together.
forming the first portion and the second portion as described herein. For instance, this step may include forming the first portion and the second portion to each comprise the first layer and the second layer.
attaching the first portion and the second portion together. For instance, this step may include attaching edge or end regions of the first portion and the second portion together.
forming the core as described herein from a core material.
positioning the core material in the cavity. For instance, this step may include injecting the core material into the cavity.

In examples, step (b) comprises:
positioning the first layer and the thermoformable layer on a shaping component. For instance, this step may include positioning the first layer and the thermoformable layer on a mandrel with a predefined shape or a mould with a predefined shape.
applying heat to the thermoformable layer. For instance, this step may include heating the shaping component.
thermo-forming the thermoformable layer. For instance, this step may include thermo-forming the thermoformable layer to the first layer.
positioning the sleeve and the core material on a shaping component with a predefined shape. For instance, this step may include positioning the sleeve and the core material in a mould with a predefined shape.
heating the core material to melt it.
allowing the melted core material to cool to harden.
allowing the core material to set or cure to harden.
allowing the core material to at least partially integrate into the second layer. For instance, this step may include melting the core material into the second layer.
moulding the sleeve and the core material into the predefined shape.

The method may further comprise the following step:
(c) removing the headgear structure from the shaping component.

In examples, step (c) may comprise:
removing the thermo-bonded thermoformable layer and the first layer from the mandrel or the mould.
removing the moulded sleeve and hardened core from the mould.

According to another aspect of the present technology there is provided a method of manufacturing a headgear structure as described herein, the method comprising the following steps, occurring in any order:
arranging at least one layer of material to form a cavity for a sleeve;
providing a core material into the cavity; and
allowing the core material to at least partially integrate into the at least one layer.

Another aspect of one form of the present technology is a patient interface that is moulded or otherwise constructed with a perimeter shape which is complementary to that of an intended wearer.

An aspect of one form of the present technology is a method of manufacturing apparatus.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a portable RPT device that may be carried by a person, e.g., around the home of the person.

An aspect of one form of the present technology is a patient interface that may be washed in a home of a patient, e.g., in soapy water, without requiring specialised cleaning equipment. An aspect of one form of the present technology is a humidifier tank that may be washed in a home of a patient, e.g., in soapy water, without requiring specialised cleaning equipment.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 3 BRIEF DESCRIPTION OF THE DRAWINGS

The present technology is illustrated by way of example, and not by way of limitation, in the figures of the accompanying drawings, in which like reference numerals refer to similar elements including:

### 3.1 Respiratory Therapy Systems

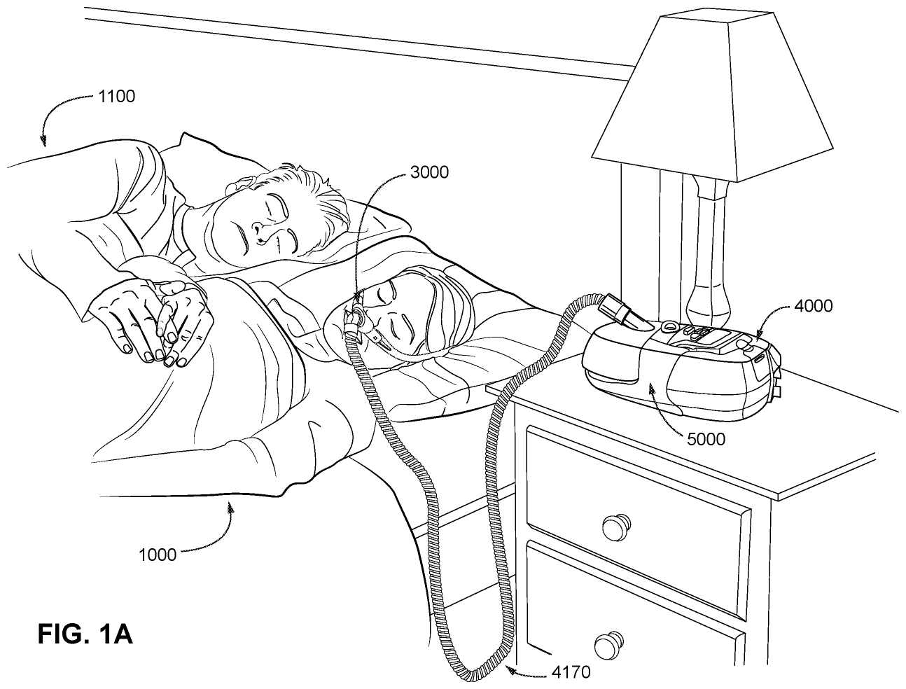

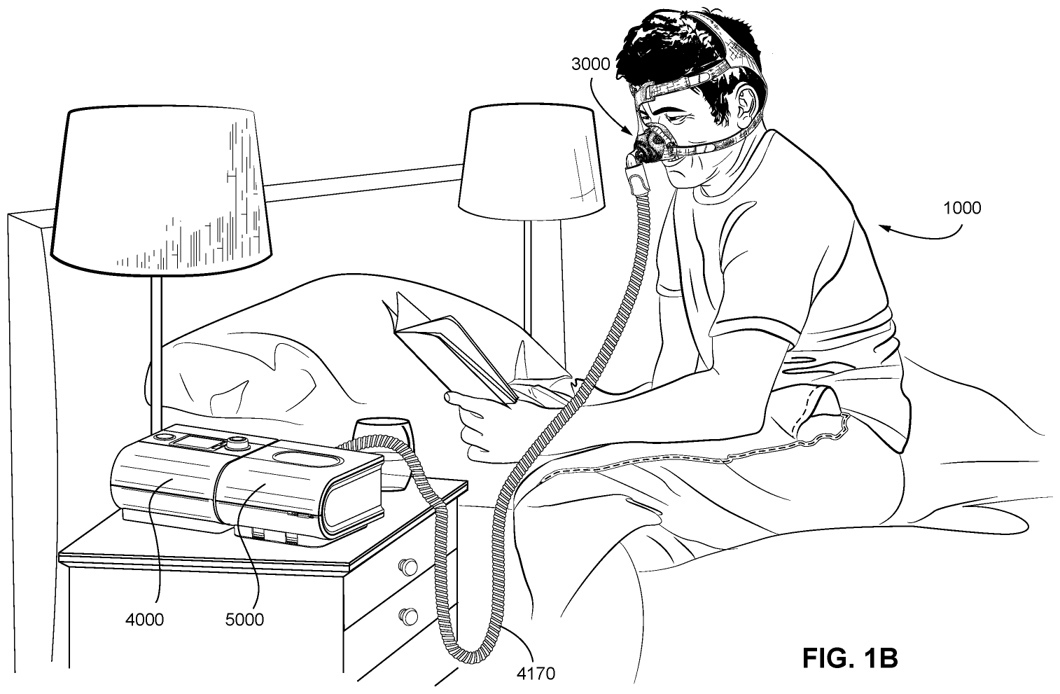

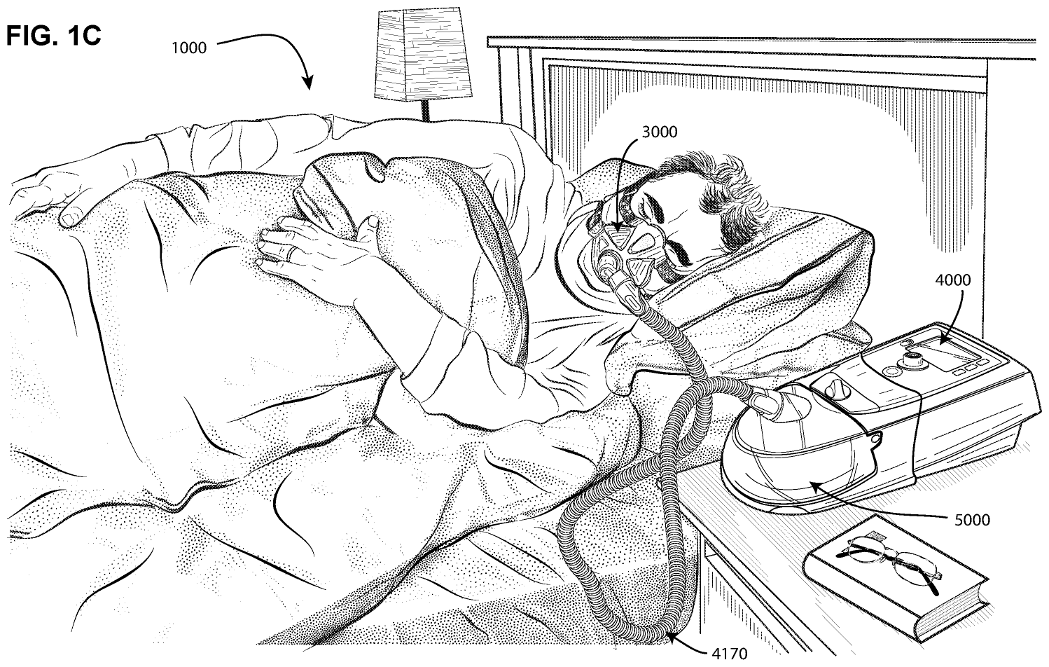

FIG. 1A shows a system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 3.2 Respiratory System and Facial Anatomy

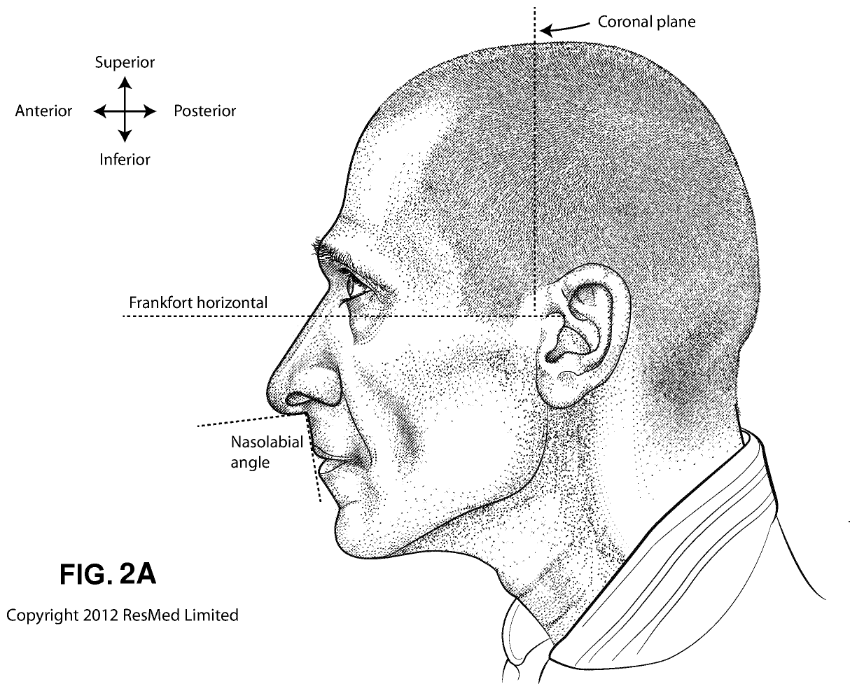

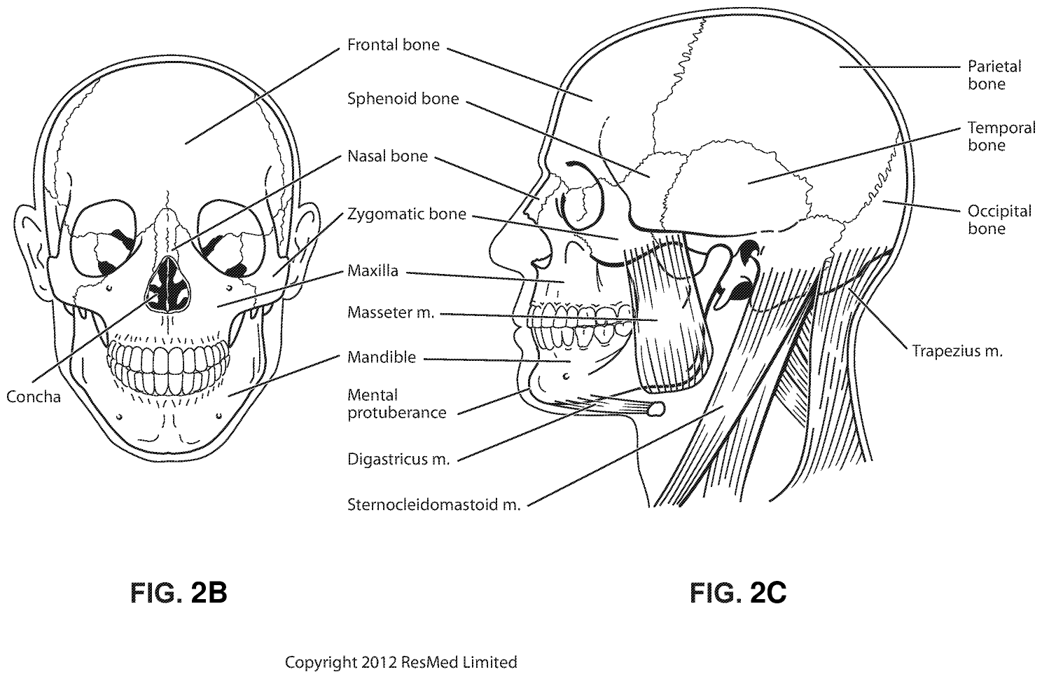

FIG. 2A is a further side view of a head. The approximate locations of the Frankfort horizontal and nasolabial angle are indicated. The coronal plane is also indicated.

FIG. 2B shows a front view of the bones of a skull including the frontal, nasal and zygomatic bones. Nasal concha are indicated, as are the maxilla, and mandible.

FIG. 2C shows a lateral view of a skull with the outline of the surface of a head, as well as several muscles. The following bones are shown: frontal, sphenoid, nasal, zygomatic, maxilla, mandible, parietal, temporal and occipital. The mental protuberance is indicated. The following muscles are shown: digastricus, masseter, sternocleidomastoid and trapezius.

### 3.3 Patient Interface

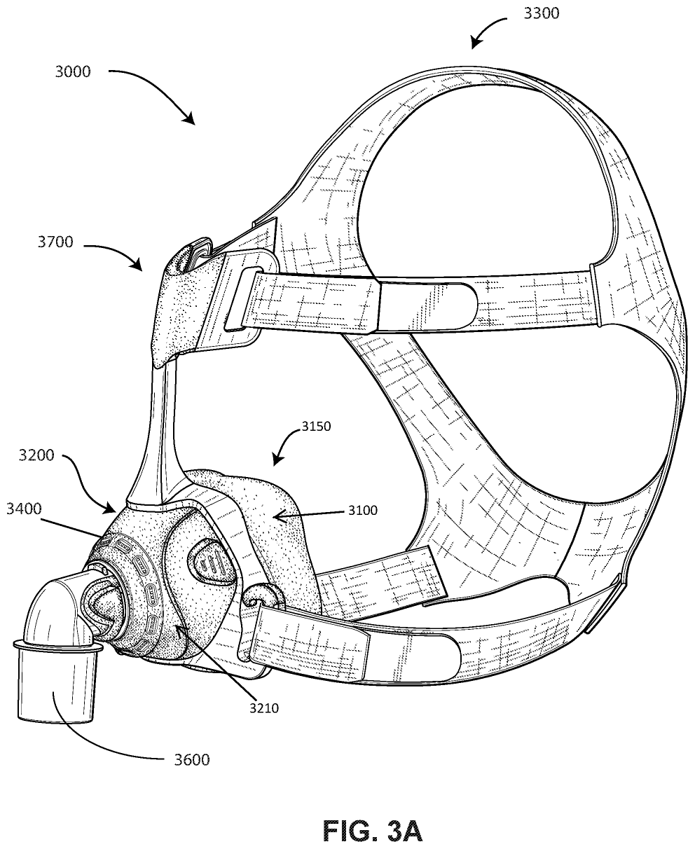

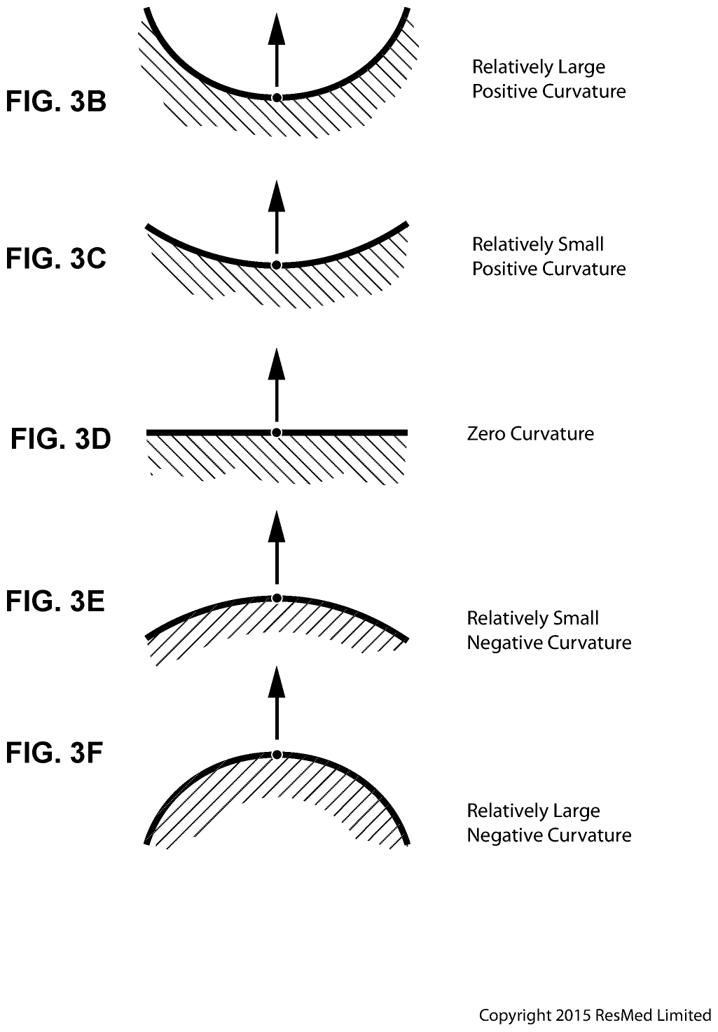

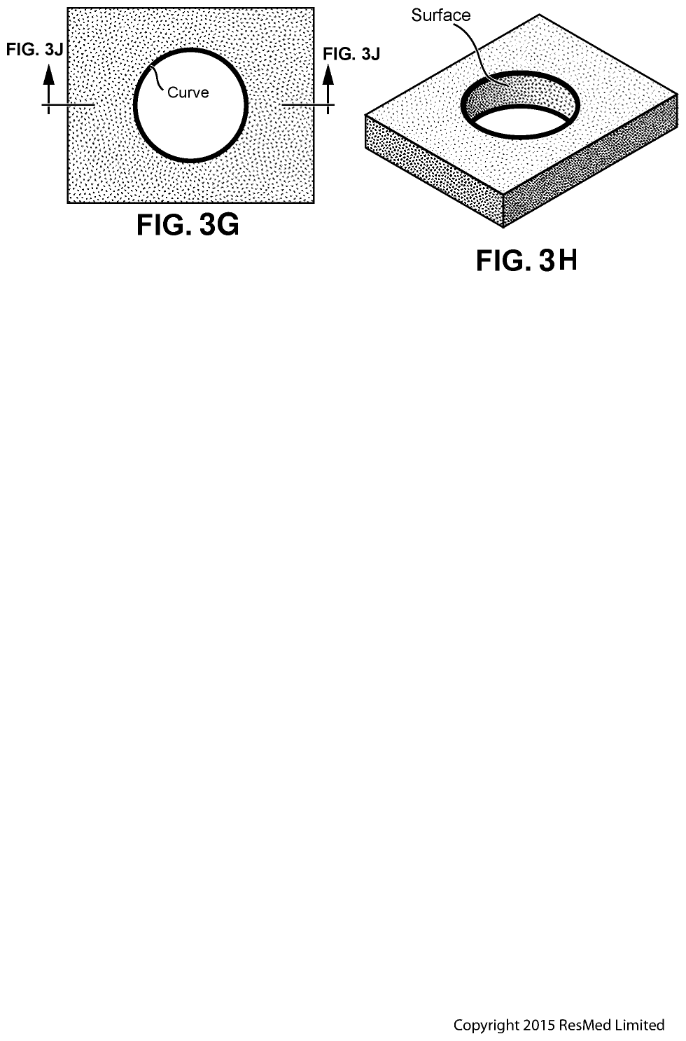

FIG. 3A shows a patient interface in the form of a nasal mask in accordance with one form of the present technology.

FIG. 3B shows a schematic of a cross-section through a structure at a point. An outward normal at the point is indicated. The curvature at the point has a positive sign, and a relatively large magnitude when compared to the magnitude of the curvature shown in FIG. 3C.

FIG. 3C shows a schematic of a cross-section through a structure at a point. An outward normal at the point is indicated. The curvature at the point has a positive sign, and a relatively small magnitude when compared to the magnitude of the curvature shown in FIG. 3B.

FIG. 3D shows a schematic of a cross-section through a structure at a point. An outward normal at the point is indicated. The curvature at the point has a value of zero.

FIG. 3E shows a schematic of a cross-section through a structure at a point. An outward normal at the point is indicated. The curvature at the point has a negative sign, and a relatively small magnitude when compared to the magnitude of the curvature shown in FIG. 3F.

FIG. 3F shows a schematic of a cross-section through a structure at a point. An outward normal at the point is indicated. The curvature at the point has a negative sign, and a relatively large magnitude when compared to the magnitude of the curvature shown in FIG. 3E.

FIG. 3G shows the surface of a structure, with a one dimensional hole in the surface. The illustrated plane curve forms the boundary of a one dimensional hole.

FIG. 3H shows a perspective view of the structure of FIG. 3G, including the two dimensional hole and the one dimensional hole. Also shown is the surface that bounds a two dimensional hole in the structure of FIG. 3G.

FIG. 3I shows a patient interface in the form of a nasal cannula in accordance with one form of the present technology.

### 3.4 RPT Device

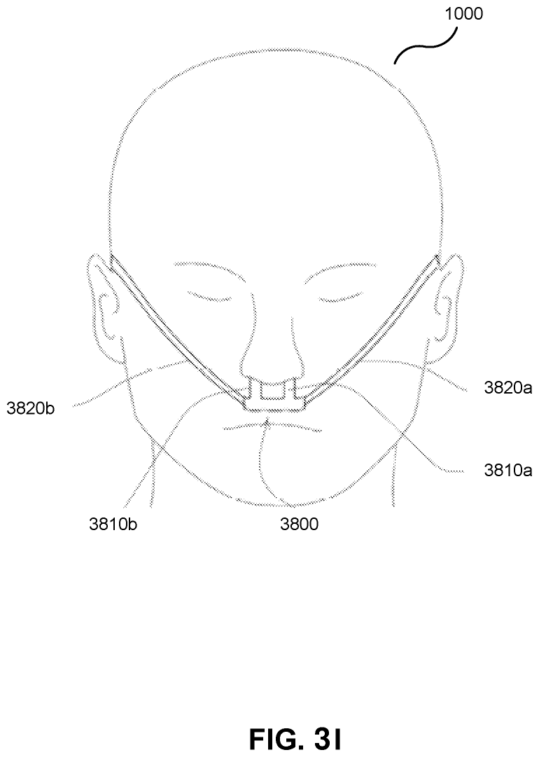

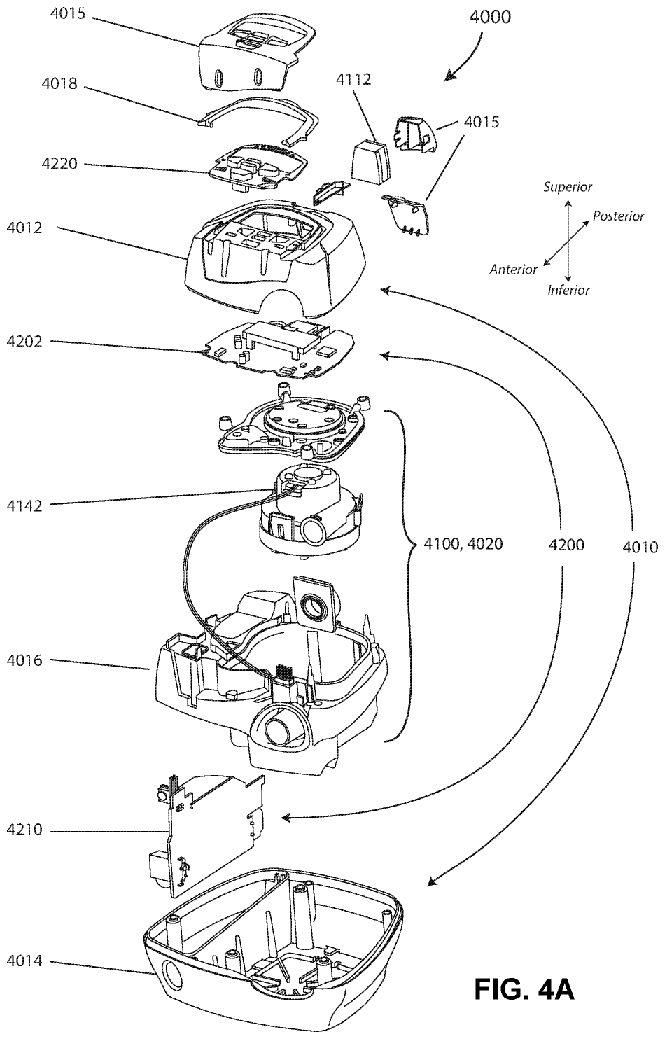

FIG. 4A shows an RPT device in accordance with one form of the present technology.

### 3.5 Humidifier

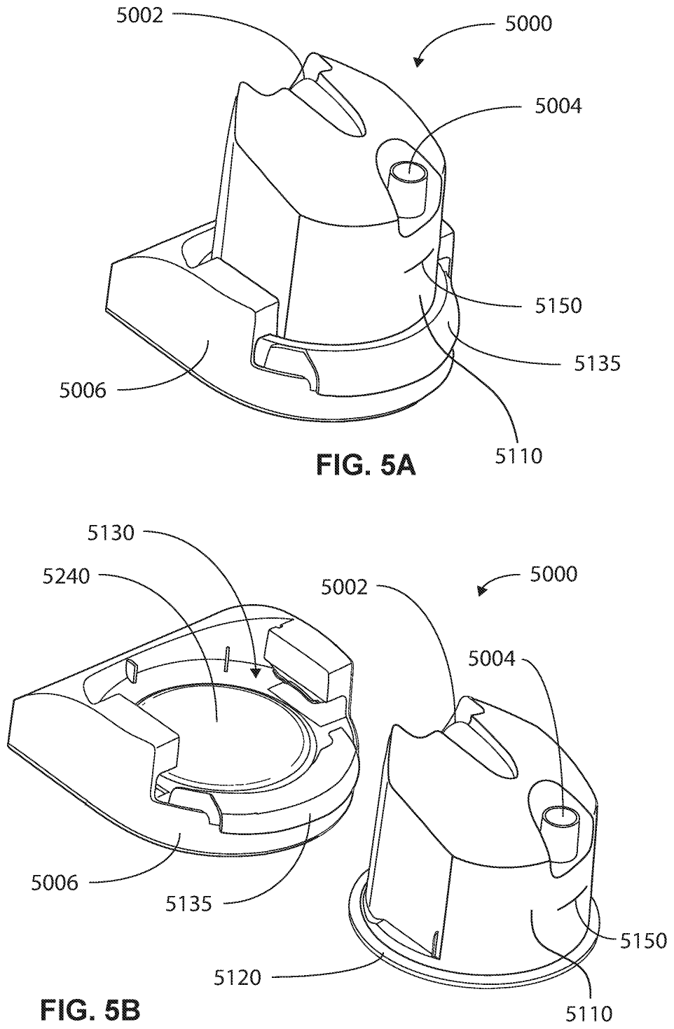

FIG. 5A shows an isometric view of a humidifier in accordance with one form of the present technology.

FIG. 5B shows an isometric view of a humidifier in accordance with one form of the present technology, showing a humidifier reservoir 5110 removed from the humidifier reservoir dock 5130.

### 3.6 First Embodiment of Headgear Structure of Positioning and Stabilising Structure

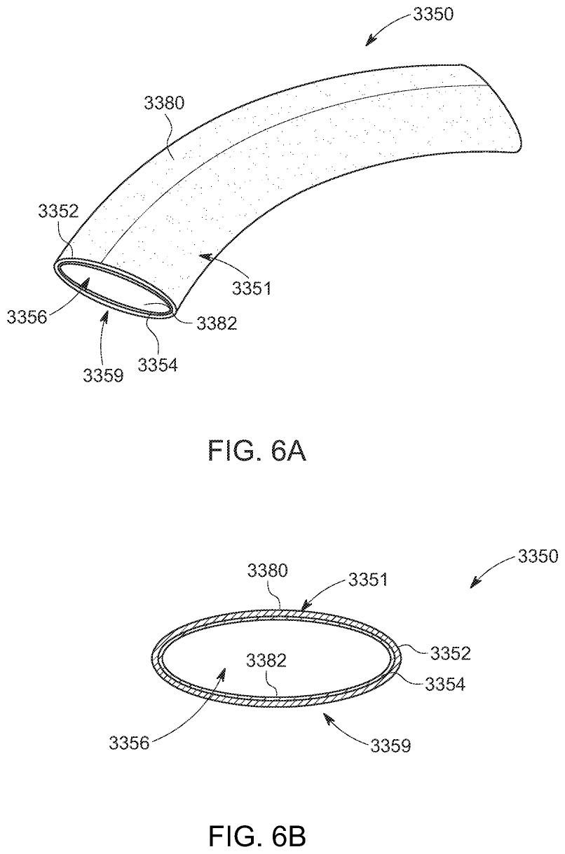

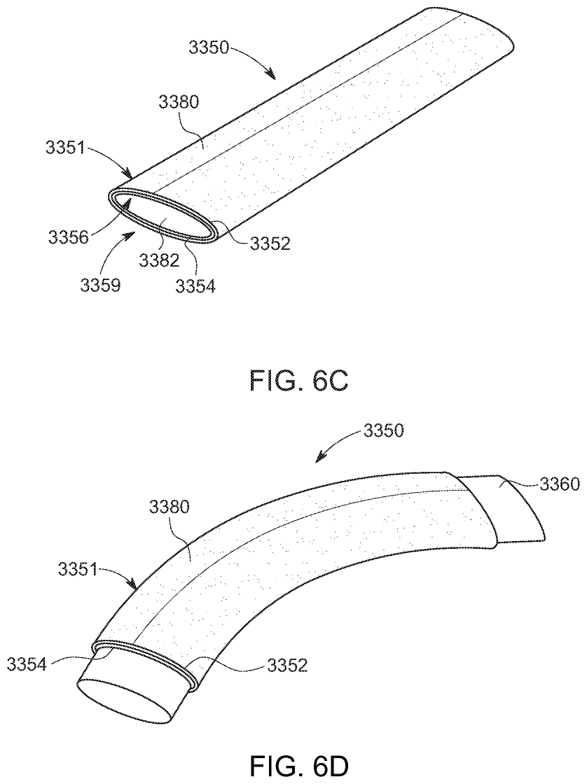

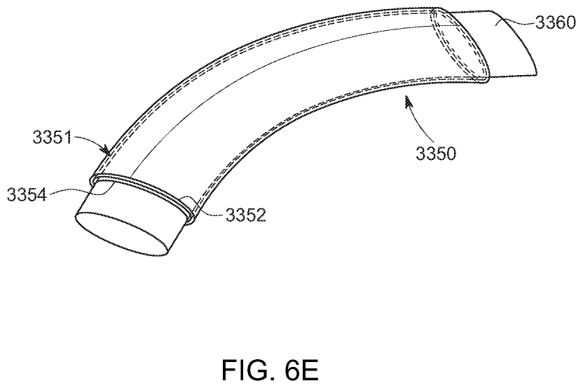

FIG. 6A shows a perspective view of a headgear structure having a predefined shape in accordance with one form of the present technology.

FIG. 6B shows a cross-sectional view through a section of the headgear structure of FIG. 6A.

FIG. 6C shows a perspective view of the headgear structure of FIG. 6A before being shaped.

FIG. 6D shows a perspective view of the headgear structure of FIG. 6C mounted on a mandrel to shape the headgear structure.

FIG. 6E shows a perspective view of the headgear structure of FIG. 6D which can be heated.

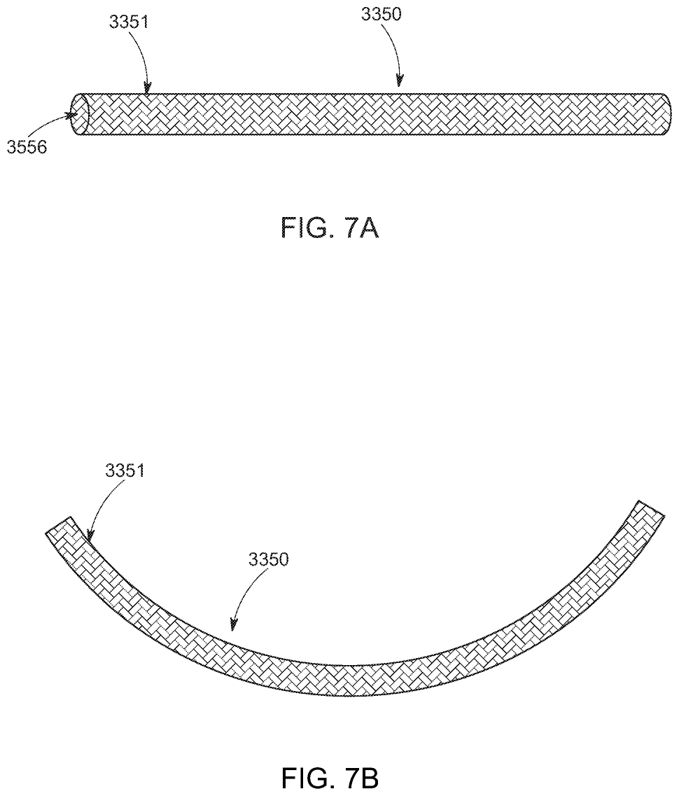

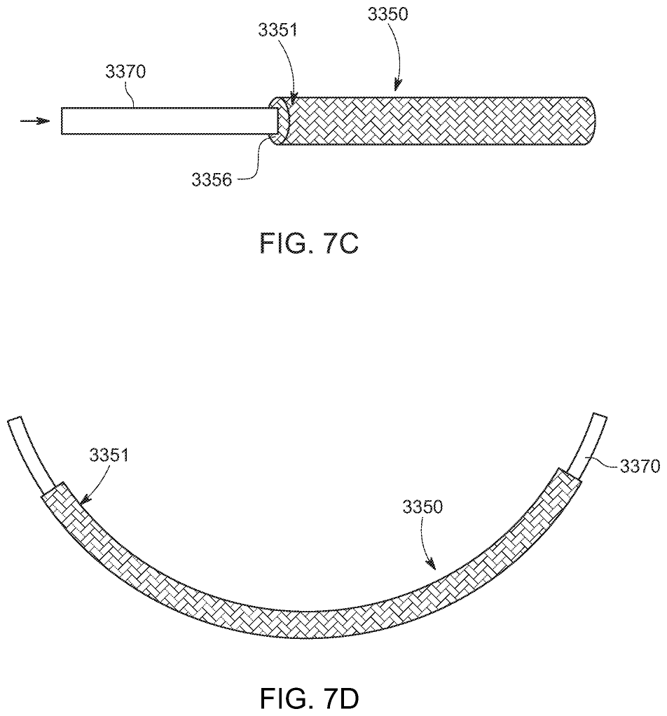

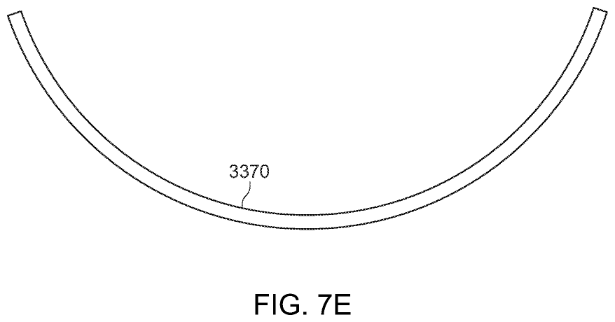

FIG. 7A shows a perspective view of a headgear structure before being shaped in accordance with one form of the present technology.

FIG. 7B shows a planar view of a headgear structure having a predefined shape in accordance with one form of the present technology.

FIG. 7C shows a perspective view of a headgear structure of FIG. 7A and represents a rigidiser component being inserted into the headgear structure in accordance with one form of the present technology.

FIG. 7D shows a planar view of a headgear structure comprising a rigidiser component in accordance with one form of the present technology.

FIG. 7E shows a planar view of a rigidiser component with a predefined shape in accordance with one form of the present technology.

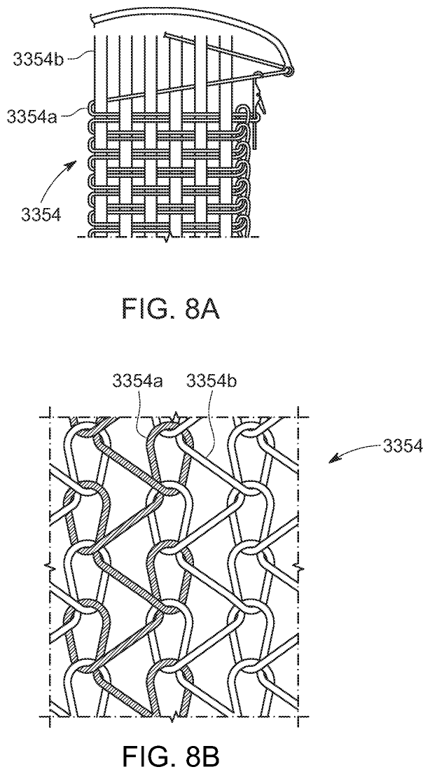

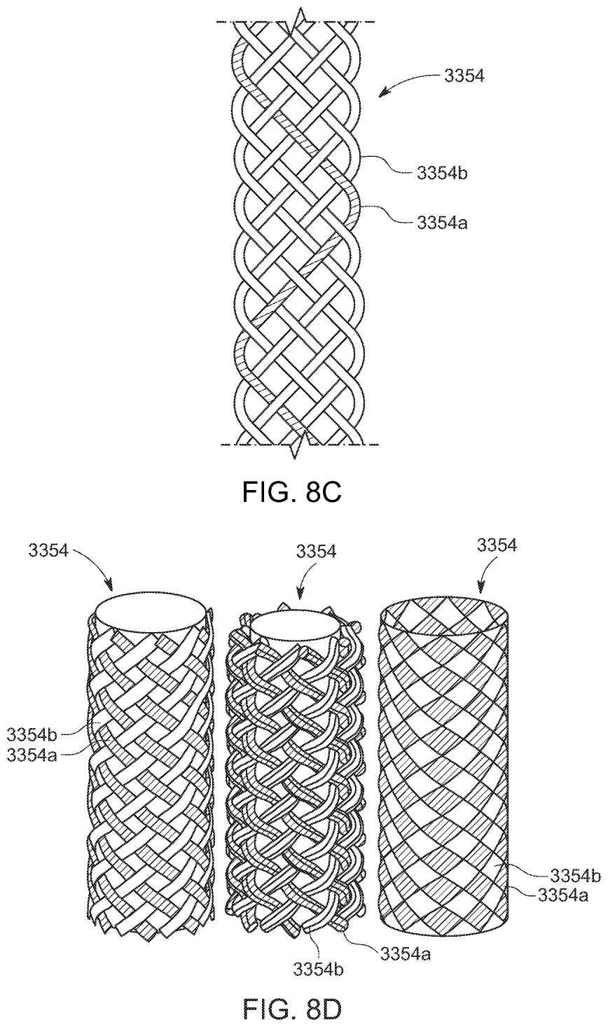

FIG. 8A shows the formation of a thermoformable layer by weaving.

FIG. 8B shows an enlarged section of a thermoformable layer formed by warping.

FIG. 8C shows an enlarged section of a thermoformable layer formed by braiding.

FIG. 8D shows a thermoformable layer formed by braiding.

### 3.7 Second Embodiment of Headgear Structure of Positioning and Stabilising Structure

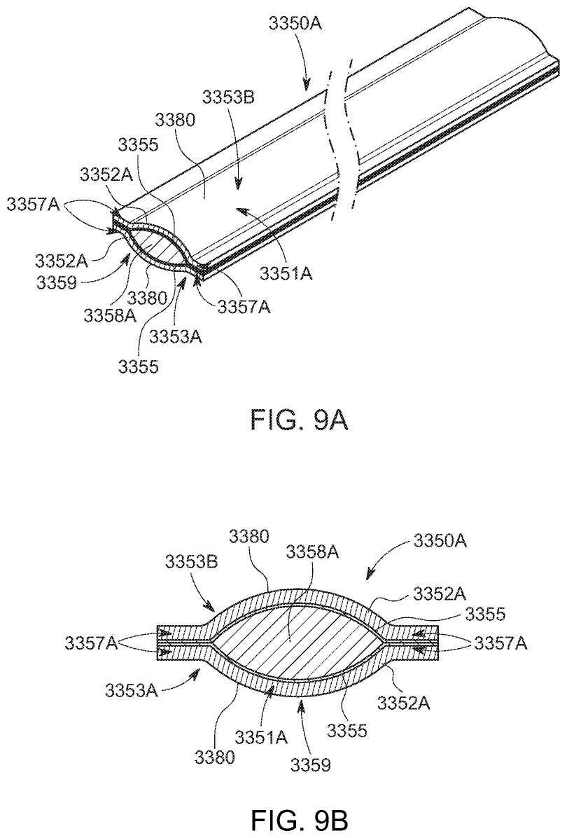

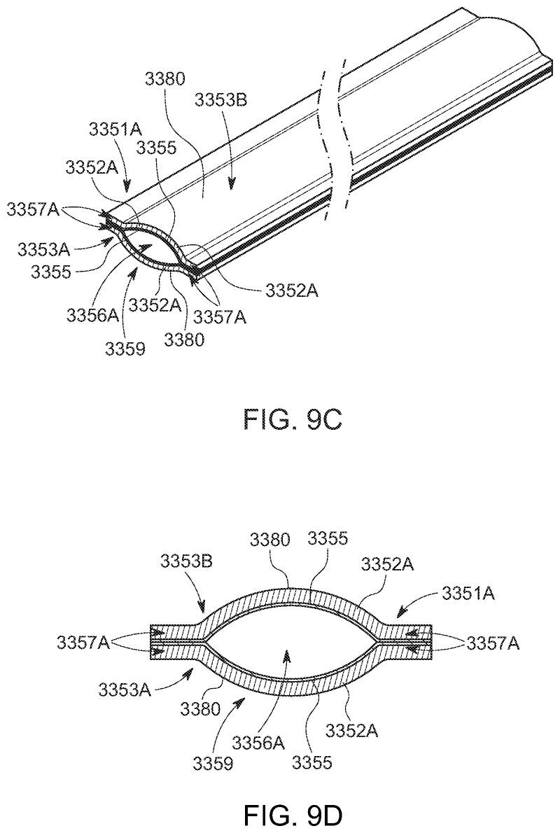

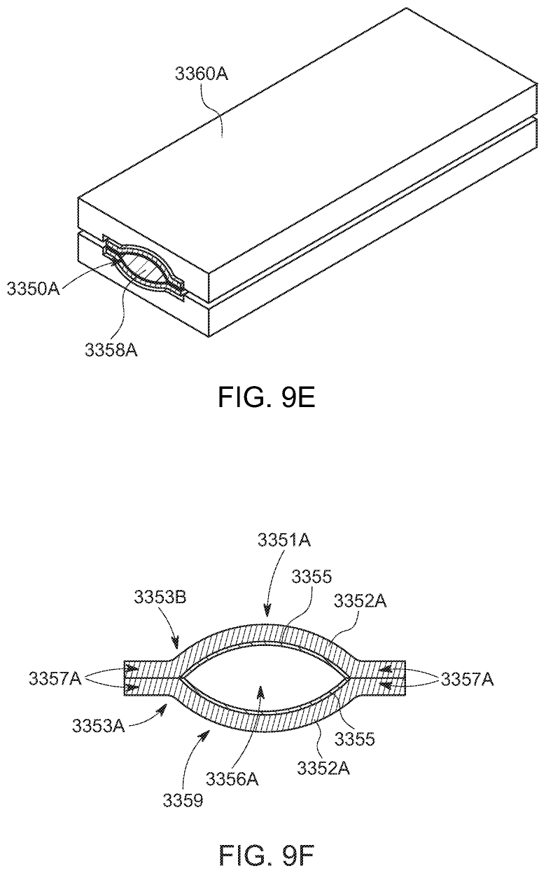

FIG. 9A shows a perspective view of a headgear structure having a predefined shape in accordance with one form of the present technology.

FIG. 9B shows a cross-sectional view through a section of the headgear structure of FIG. 9A.

FIG. 9C shows a perspective view if the headgear structure of FIG. 9A in the form of a sleeve, and before forming a core inside the sleeve and shaping the headgear structure.

FIG. 9D shows a cross-sectional view through a section of the headgear structure of FIG. 9C.

FIG. 9E shows a perspective view of the headgear structure of FIG. 9A in a mould to shape the headgear structure.

FIG. 9F shows a cross-sectional view through a section of a headgear structure having a predefined shape in accordance with one form of the present technology.

### 4 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 4.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 4.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000 or 3800.

### 4.3 Patient Interface

A non-invasive patient interface 3000 in accordance with one aspect of the present technology is illustrated in FIG. 3A, and comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

An unsealed patient interface 3800 in accordance with one aspect of the present technology is illustrated in FIG. 31, in the form of a nasal cannula, includes nasal prongs 3810a, 3810b which can deliver air to respective nares of the patient 1000 via respective orifices in their tips. Such nasal prongs do not generally form a seal with the inner or outer skin surface of the nares. This type of interface results in one or more gaps that are present in use by design (intentional) but they are typically not fixed in size such that they may vary unpredictably by movement during use. This can present a complex pneumatic variable for a respiratory therapy system when pneumatic control and/or assessment is implemented, unlike other types of mask-based respiratory therapy systems. The air to the nasal prongs may be delivered by one or more air supply lumens 3820a, 3820b that are coupled with the nasal cannula-type unsealed patient interface 3800. The lumens 3820a, 3820b lead from the nasal cannula-type unsealed patient interface 3800 to a respiratory therapy device via an air circuit. The unsealed patient interface 3800 is particularly suitable for delivery of flow therapies, in which the RPT device generates the flow of air at controlled flow rates rather than controlled pressures. The “vent” or gap at the unsealed patient interface 3800, through which excess airflow escapes to ambient, is the passage between the end of the prongs 3810a and 3810b of the nasal cannula-type unsealed patient interface 3800 via the patient's nares to atmosphere.

A patient interface 3000, 3800 in accordance with one aspect of the present technology comprises a positioning and stabilising structure 3300. The positioning and stabilising structure 3300 may comprise at least one of a headgear structure 3350 and a headgear structure 3350A. For example, the headgear structure 3350, 3350A may be formed as part of a headgear assembly, e.g. the headgear assembly shown in FIG. 3A.

In some forms, the headgear structure 3350, 3350A may be formed in a predefined shape, e.g. a 3D shape. For example, the headgear structure 3350, 3350A may be formed with a 3D shape over the length and/or width of the headgear structure 3350, 3350A. For example, at least a portion of the headgear structure 3350, 3350A may curve in one or more directions.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure of at least 6 cmH2O with respect to ambient.

The patient interface 3000 in accordance with one form of the present technology is constructed and arranged to be able to provide a supply of air at a positive pressure of at least 10 cmH2O with respect to ambient.

The patient interface 3000 in accordance with one form of the present technology is constructed and arranged to be able to provide a supply of air at a positive pressure of at least 20 cmH2O with respect to ambient.

### 4.3.1 Seal-Forming Structure

In some examples of the present technology a patient interface 3000 may comprise a seal-forming structure 3100 configured to form a seal with a region of the patient's face surrounding one or more entrance to the patient's airways. The seal-forming structure 3100 may be configured to assist with maintaining a therapeutic pressure in the plenum chamber 3200 throughout the patient's respiratory cycle in use.

In some forms, the seal-forming structure 3100 is configured to form a seal with or around portions of the patient's nose in use. For example, the seal-forming structure 3100 is configured to form a seal in use with the underside of the patient's nose, around the patient's nares.

The seal-forming structure 3100 may comprise at least one hole, or a pair of holes, neither of which are shown in the figures, but which may be configured to allow a flow of air at therapeutic pressure to be delivered to the patient's nares. Each hole aligns in use with a respective nare of the patient. In some examples, the at least one hole is formed in a central portion of the seal-forming structure 3100.

In other forms which are not illustrated, the seal-forming structure 3100 may be configured to form a seal in use with the other parts of patient's face, e.g. with or around portions of the patient's nose and with or around portions of the patient's mouth. For example, the seal-forming structure 3100 may comprise a hole which may be configured to allow a flow of air at therapeutic pressure to be delivered to the patient's mouth. Therefore, it is to be appreciated that in some forms the patient interface 3000 may be a full face or oro-nasal mask and is not limited to a nasal mask.

In one form of the present technology, a seal-forming structure 3100 provides a target seal-forming region, and may additionally provide a cushioning function. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 3100.

In certain forms of the present technology, the seal-forming structure 3100 is constructed from a biocompatible material, e.g. silicone rubber.

A seal-forming structure 3100 in accordance with the present technology may be constructed from a soft, flexible, resilient material such as silicone.

In certain forms of the present technology, a system is provided comprising more than one a seal-forming structure 3100, each being configured to correspond to a different size and/or shape range. For example the system may comprise one form of a seal-forming structure 3100 suitable for a large sized head, but not a small sized head and another suitable for a small sized head, but not a large sized head.

### 4.3.1.1 Sealing Mechanisms

In one form, the seal-forming structure includes a sealing flange utilizing a pressure assisted sealing mechanism. In use, the sealing flange can readily respond to a system positive pressure in the interior of the plenum chamber 3200 acting on its underside to urge it into tight sealing engagement with the face. The pressure assisted mechanism may act in conjunction with elastic tension in the positioning and stabilising structure.

In one form, the seal-forming structure 3100 comprises a sealing flange and a support flange. The sealing flange comprises a relatively thin member with a thickness of less than about 1 mm, for example about 0.25 mm to about 0.45 mm, which extends around the perimeter of the plenum chamber 3200. Support flange may be relatively thicker than the sealing flange. The support flange is disposed between the sealing flange and the marginal edge of the plenum chamber 3200, and extends at least part of the way around the perimeter. The support flange is or includes a spring-like element and functions to support the sealing flange from buckling in use.

In one form, the seal-forming structure may comprise a compression sealing portion or a gasket sealing portion. In use the compression sealing portion, or the gasket sealing portion is constructed and arranged to be in compression, e.g. as a result of elastic tension in the positioning and stabilising structure.

In one form, the seal-forming structure comprises a tension portion. In use, the tension portion is held in tension, e.g. by adjacent regions of the sealing flange.

In one form, the seal-forming structure comprises a region having a tacky or adhesive surface.

In certain forms of the present technology, a seal-forming structure may comprise one or more of a pressure-assisted sealing flange, a compression sealing portion, a gasket sealing portion, a tension portion, and a portion having a tacky or adhesive surface.

### 4.3.1.2 Nose Bridge or Nose Ridge Region

In one form, the non-invasive patient interface 3000 comprises a seal-forming structure that forms a seal in use on a nose bridge region or on a nose-ridge region of the patient's face.

In one form, the seal-forming structure includes a saddle-shaped region constructed to form a seal in use on a nose bridge region or on a nose-ridge region of the patient's face.

### 4.3.1.3 Upper Lip Region

In one form, the non-invasive patient interface 3000 comprises a seal-forming structure that forms a seal in use on an upper lip region (that is, the lip superior) of the patient's face.

In one form, the seal-forming structure includes a saddle-shaped region constructed to form a seal in use on an upper lip region of the patient's face.

### 4.3.1.4 Chin-Region

In one form the non-invasive patient interface 3000 comprises a seal-forming structure that forms a seal in use on a chin-region of the patient's face.

In one form, the seal-forming structure includes a saddle-shaped region constructed to form a seal in use on a chin-region of the patient's face.

### 4.3.1.5 Forehead Region

In one form, the seal-forming structure that forms a seal in use on a forehead region of the patient's face. In such a form, the plenum chamber may cover the eyes in use.

### 4.3.1.6 Nasal Pillows

In one form the seal-forming structure of the non-invasive patient interface 3000 comprises a pair of nasal puffs, or nasal pillows, each nasal puff or nasal pillow being constructed and arranged to form a seal with a respective naris of the nose of a patient.

Nasal pillows in accordance with an aspect of the present technology include: a frusto-cone, at least a portion of which forms a seal on an underside of the patient's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

### 4.3.2 Plenum Chamber

A patient interface 3000 according to some examples of the present technology comprises a plenum chamber 3200 pressurisable to a therapeutic pressure of at least 6 cmH2O above ambient air pressure. The plenum chamber 3200 may receive a flow of air at the therapeutic pressure for breathing by a patient.

The plenum chamber 3200 in some forms of the present technology is at least partially provided by a cushion module 3150 of the patient interface 3000.

As is perhaps best illustrated in FIG. 3A, the cushion module 3150 according to some examples may comprise a frame portion 3210 and the seal-forming structure 3100.

The plenum chamber 3200 may be at least partially formed by both the frame portion 3210 and the seal-forming structure 3100. The frame portion 3210 may support the seal-forming structure 3100 in position against the patient's face in use. The frame portion 3210 and seal-forming structure 3100 may together partially enclose a volume of space which in use has air therein pressurised above atmospheric pressure, forming the plenum chamber 3200.

In particular, the frame portion 3210 may at least partially define part of the plenum chamber 3200 pressurisable to a therapeutic pressure of at least 6 cmH2O above ambient air pressure.

The seal-forming structure 3100 may be provided to the frame portion 3210 and may at least partially form the plenum chamber 3200. The seal-forming structure 3100 may be connected to the frame portion 3210, either permanently connected or removably connected.

In certain forms, the plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200.

In some forms, the plenum chamber 3200 is formed from a single homogeneous piece of material. In some forms, the plenum chamber 3200 may be formed from a homogenous piece of material fitted with connectors formed from another material. In other forms, the plenum chamber 3200 is constructed from a plurality of materials, for example one material may be used to form the frame portion 3210 and another material may be used to form the seal-forming structure 3100, with the plenum chamber 3200 comprising at least a part of both the frame portion 3210 and the seal-forming structure 3100.

In certain forms of the present technology, the plenum chamber 3200 does not cover the eyes of the patient in use. In other words, the eyes are outside the pressurised volume defined by the plenum chamber 3200. Such forms tend to be less obtrusive and/or more comfortable for the wearer, which can improve compliance with therapy.

In certain forms of the present technology, the plenum chamber 3200 is constructed from a transparent material, e.g. silicone, a thermoplastic elastomer, a transparent polycarbonate or the like. For example, the majority of the cushion module 3150 can be formed from silicone. In particular, the frame portion 3210 and seal-forming structure 3100 are both formed from silicone in those examples. The use of a transparent material can reduce the obtrusiveness of the patient interface 3000, and can help improve compliance with therapy. The use of a transparent material can aid a clinician to observe how the patient interface 3000 is located and functioning.

In certain forms of the present technology, the plenum chamber 3200 is constructed from a translucent material. The use of a translucent material can reduce the obtrusiveness of the patient interface 3000, and can help improve compliance with therapy.

In the exemplary forms of the technology, the frame portion 3210 may be flexible, for example it may be formed from a material having a relatively low modulus of elasticity. The seal-forming structure 3100 may also be flexible, for example formed from the same material, or another material having a relatively low modulus of elasticity. In some examples, the frame portion 3210 and seal-forming structure 3100 are integrally formed and may be formed from the deformable material. The frame portion 3210 may be formed from an elastomeric material, such as silicone. The seal-forming structure 3100 may be formed from the elastomeric material. The frame portion 3210 and seal-forming structure 3100 may comprise one piece. In these examples, the frame portion 3210 and seal-forming structure 3100 are moulded together as a single part formed from an elastomeric material, e.g. silicone. The seal-forming structure 3100 and the frame portion 3210 (or at least a majority of the frame portion 3210) may be formed (e.g. constructed, moulded or the like) together from a single homogenous piece of a deformable material, such as an elastomeric material, e.g. silicone. The frame portion 3210 and the seal-forming structure 3100 may be of unitary construction.

In other examples of the technology the seal-forming structure 3100 may be removably connected to the frame portion 3210. The seal-forming structure 3100 may be connected to the frame portion 3210 by a soft-to-soft, soft-to-hard or a hard-to-hard connection.

In the form of the present technology, the plenum chamber 3200 comprises at least one inlet (not shown) configured to receive a flow of air at the therapeutic pressure for breathing by the patient. For example, the inlet(s) may be provided in any suitable location on the plenum chamber, e.g. on one of more of an anterior portion and lateral portion of the plenum chamber 3200.

In some examples, at least one rigidiser may be provided to the frame portion 3210 to make it more rigid (while still being flexible). In some examples, the frame portion 3210 and the seal-forming structure 3100 are formed from a material having a relatively low modulus of elasticity (e.g. silicone, TPE or the like) and the frame portion 3210 comprises the rigidiser(s). In particular, the patient interface 3000 or cushion module 3150 may comprise the rigidiser(s). The rigidiser(s) may be provided to the frame portion 3210. The rigidiser(s) may be configured to rigidise the frame portion 3210. The rigidiser(s) may be configured to resist deformation of the frame portion 3210. The rigidiser(s) may be configured to provide support to the frame portion 3210.

The rigidiser(s) may be provided by one or more of: a thickened region, a structure or component which is rigid relative to the frame portion and which is one of removably attached, permanently attached, and integrally formed with the frame portion 3210.

### 4.3.3 Positioning and Stabilising Structure

The seal-forming structure 3100 of the patient interface 3000 of the present technology may be held in sealing position in use by the positioning and stabilising structure 3300.

In one form the positioning and stabilising structure 3300 provides a retention force at least sufficient to overcome the effect of the positive pressure in the plenum chamber 3200 to lift off the face.

In one form the positioning and stabilising structure 3300 provides a retention force to overcome the effect of the gravitational force on the patient interface 3000.

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus. In one example, the positioning and stabilising structure 3300 comprises at least one strap having a rectangular cross-section. In one example the positioning and stabilising structure 3300 comprises at least one flat strap. In another example, the positioning and stabilising structure 3300 comprises at least one strap having a non-rectangular cross-section, e.g. a substantially oval-shaped cross-section or a substantially lens-shaped cross-section.

In one form of the present technology, a positioning and stabilising structure 3300 is provided that is configured so as not to be too large and bulky to prevent the patient from lying in a supine sleeping position with a back region of the patient's head on a pillow.

In one form of the present technology, a positioning and stabilising structure 3300 is provided that is configured so as not to be too large and bulky to prevent the patient from lying in a side sleeping position with a side region of the patient's head on a pillow.

In one form of the present technology, a positioning and stabilising structure 3300 is provided with a decoupling portion located between an anterior portion of the positioning and stabilising structure 3300, and a posterior portion of the positioning and stabilising structure 3300. The decoupling portion does not resist compression and may be, e.g. a flexible or floppy strap. The decoupling portion is constructed and arranged so that when the patient lies with their head on a pillow, the presence of the decoupling portion prevents a force on the posterior portion from being transmitted along the positioning and stabilising structure 3300 and disrupting the seal.

In one form of the present technology, a positioning and stabilising structure 3300 comprises a strap constructed from a laminate of a fabric patient-contacting layer, a foam inner layer and a fabric outer layer. In one form, the foam is porous to allow moisture, (e.g., sweat), to pass through the strap. In one form, the fabric outer layer comprises loop material to engage with a hook material portion.

In certain forms of the present technology, a positioning and stabilising structure 3300 comprises a strap that is extensible, e.g. resiliently extensible. For example the strap may be configured in use to be in tension, and to direct a force to draw a seal-forming structure into sealing contact with a portion of a patient's face. In an example the strap may be configured as a tie.

In one form of the present technology, the positioning and stabilising structure comprises a first tie, the first tie being constructed and arranged so that in use at least a portion of an inferior edge thereof passes superior to an otobasion superior of the patient's head and overlays a portion of a parietal bone without overlaying the occipital bone.

In one form of the present technology suitable for a nasal-only mask or for a full-face mask, the positioning and stabilising structure includes a second tie, the second tie being constructed and arranged so that in use at least a portion of a superior edge thereof passes inferior to an otobasion inferior of the patient's head and overlays or lies inferior to the occipital bone of the patient's head.

In one form of the present technology suitable for a nasal-only mask or for a full-face mask, the positioning and stabilising structure includes a third tie that is constructed and arranged to interconnect the first tie and the second tie to reduce a tendency of the first tie and the second tie to move apart from one another.

In certain forms of the present technology, a positioning and stabilising structure 3300 comprises a strap that is bendable and e.g. non-rigid. An advantage of this aspect is that the strap is more comfortable for a patient to lie upon while the patient is sleeping.

In certain forms of the present technology, a positioning and stabilising structure 3300 comprises a strap constructed to be breathable to allow moisture vapour to be transmitted through the strap,

In certain forms of the present technology, a system is provided comprising more than one positioning and stabilizing structure 3300, each being configured to provide a retaining force to correspond to a different size and/or shape range. For example the system may comprise one form of positioning and stabilizing structure 3300 suitable for a large sized head, but not a small sized head, and another. suitable for a small sized head, but not a large sized head.

In some forms, the positioning and stabilising structure 3300 comprises the headgear structure 3350, 3350A. The headgear structure 3350, 3350A may be configured to extend, in use, over a portion of the patient's face and/or head.

In some forms, the headgear structure 3350, 3350A may comprise an outer surface 3380 which contacts the patient's face and/or head in use.

In some forms, the headgear structure 3350, 3350A may comprise a headgear strap or tie. That is, the headgear structure 3350, 3350A may be formed into a headgear strap or tie.

### 4.3.3.1 First Embodiment of Headgear Structure

Referring now to FIGS. 6A to 6E which illustrate a first embodiment of a headgear structure 3350 according to an aspect of the present technology.

In some forms, as shown in FIG. 6A, for example, the headgear structure may be hollow. That is, at least a portion of the headgear structure 3350 along its length may be hollow. This will be described in more detail further below.

In some forms, the headgear structure 3350 may comprise an inner surface 3382. The inner surface 3382 may be configured to define a cavity 3356 in the headgear structure 3350. The inner surface 3382 may surround the cavity 3356 which extends through at least a portion of the length of the headgear structure 3350. This may create a hollow headgear structure 3350.

The headgear structure 3350 may comprise a sleeve 3351. The sleeve 3351 may be formed from at least one layer of material. The sleeve 3351 may be a continuous structure. The sleeve 3351 may have an outer surface which is continuous. This may provide the outer surface 3380 of the headgear structure 3350 which is continuous. This will also be described in more detail below.

In some forms, the headgear structure 3350 comprises at least one layer. In a preferred form, the headgear structure 3350 comprises a plurality of layers.

In some forms, the sleeve 3351 comprises at least a first layer 3352. In some forms, the first layer 3352 is a single layer of material, however it may be formed by a plurality of layers or sub-layers. For example, the first layer 3352 may comprise a polyamide material, e.g. nylon, and/or a polyurethane material, e.g. a polyurethane co-polymer material such as elastane (e.g. spandex, Lycra®, or ROICA™). In one form, the first layer 3352 may comprise polyamide material, e.g. 80% to 95% nylon, and a polyurethane material, e.g. 5% to 20% spandex, Lycra®, or ROICA™.

In some forms, at least one of the layers may provide a patient contacting portion 3359 of the headgear structure 3350 which contacts, in use, the patient's skin and/or hair. For example, as shown, in some forms, the first layer 3352 provides the patient contacting portion 3359. In such forms, the first layer 3352 forms an outer layer of the headgear structure 3350. Therefore, the first layer 3352, which forms the sleeve 3351, has an outer surface which forms the outer surface 3380 of the headgear structure 3350 which contacts the patient's face and/or head in use.

At least one of the layers may be substantially soft. For example, the first layer 3352 may comprise soft material. This may improve patient comfort and provides the headgear structure 3350 with a soft touch or feel.

In some forms, at least one of the layers may be substantially breathable. For example, the first layer 3352 may comprise breathable material. This may improve moisture control and promote moisture wicking.

In some forms, at least one of the layers may be substantially elastic. For example, the first layer 3352 may comprise an elastic material. This can allow the first layer 3352 to stretch in at least one direction.

In some forms, the headgear structure 3350 may comprise a thermoformable layer 3354. The thermoformable layer 3354 may act as a rigidiser. The thermoformable layer 3354 may be thermoformed to at least one of the layers that form the sleeve 3351 to provide a pre-defined shape to the sleeve 3351.

In some forms, the thermoformable layer 3354 may comprise a thermoformable material. As is perhaps best illustrated in FIG. 6B, the thermoformable layer 3354 provides the headgear structure 3350 with a second layer. The second layer may provide an inner layer of the headgear structure 3350. That is, it may be positioned radially inwards with respect to the first layer 3352 which forms the outer layer of the headgear structure 3350.

In some forms, the thermoformable layer 3354 may be thinner than the first layer 3352, as shown in FIG. 6B. That is, the dimensions between an inner surface and an outer surface of the thermoformable layer 3354 are smaller or less than the dimensions between an inner surface and an outer surface of the first layer 3352. However, in other forms not shown, the thermoformable layer 3354 and the first layer 3352 may have substantially the same thickness, or the thermoformable layer 3354 may be thicker than the first layer 3352.

In some forms, the thermoformable layer 3354 may be thermoformed to at least one other layer of the headgear structure 3350 to provide a pre-defined shape to the headgear structure. For example, as will be discussed in more detail below, the thermoformable layer 3354 is thermoformed to the first layer 3352 to provide a pre- defined shape to the headgear structure 3350. As described above, in some forms such as in the embodiment illustrated, the first layer 3352 forms the sleeve 3351 or part of it, therefore, the thermoformable layer 3354 may be thermoformed to an inner surface of the sleeve 3351.

In some forms, the thermoformable layer 3354 may be substantially elastic. For example, the thermoformable layer 3354 may comprise an elastic material. This can allow the thermoformable layer 3354 to stretch in at least one direction.

The thermoformable layer 3354 may be positioned against at least a portion of one of the other layers of the headgear structure 3350. For example, the thermoformable layer 3354 may be positioned with respect to the first layer 3352 so that it extends over at least a portion of an inner surface of the first layer 3352, e.g. extending over the entire inner surface. Therefore, the thermoformable layer 3354 may form the inner layer of the headgear structure 3350 and the first layer 3352 may form the outer layer or the patient contacting portion 3359 of the headgear structure 3350. In some forms, the thermoformable layer 3354 may be positioned on the inner surface of the sleeve 3351 such that the thermoformable layer 3354 covers the entire inner surface of the sleeve 3351 to form the inner surface of the headgear structure 3350.

In some forms, the headgear structure 3350 is configured to reduce, limit (or minimize) and/or prevent the formation of sharp or uneven edges, seams and/or joints on the outer surface 3380. For example, the headgear structure 3350 does not have any sharp or uneven edges, seams and/or joints on the outer surface 3380 of the headgear structure 3350. In other words, the headgear structure 3350 has a substantially smooth outer surface 3380. FIGS. 6A to 6E show a line extending along the length of the outer surface 3380 of the headgear structure 3350. This is not a joint, and has been included for illustrative purposes to demonstrate that the headgear structure 3350 has a 3D shape.

In some forms, the headgear structure 3350 may be formed in a continuous sleeve structure, as illustrated in FIG. 6B. Therefore, the headgear structure 3350 may be substantially seamless, joint-free, edge-free, and/or smooth. This may limit or prevent irritation and/or the formation of marks on the patient's face and/or head when using the patient interface 3000, and may improve patient comfort and fit leading to increased compliance to therapy.

As mentioned above, in some forms, the outer surface of the sleeve 3351 may be continuous. The outer surface of the sleeve 3351 may be configured to be smooth, seamless and/or joint free. When viewed in cross-section from a side in a direction substantially parallel to a longitudinal axis of the headgear structure 3350, as shown in FIG. 6B, the sleeve 3351 may be a continuous structure,

In some forms, the inner surface of the sleeve 3351 may be continuous. The inner surface of the sleeve 3351 may be configured to be smooth, seamless and/or joint free.

In some forms, the headgear structure 3350 has a substantially oval-shaped cross-section, as illustrated. However, the cross-section may be any other suitable shape that does not create sharp or uneven edges, seams and/or joints, e.g. obround, circular or polygonal with substantially rounded corners or edges. In some forms, the headgear structure 3350 comprises a patient facing side and a non-patient facing side. In some forms, as shown, at least one of the patient facing side and the non-patient facing side may be substantially convex shaped. In some forms, as shown, the boundary or junction between the patient facing side and the non-patient facing side of the headgear structure 3350 may be substantially convex-shaped.

In some forms, at least one of the layers of the headgear structure 3350 is provided in a continuous sleeve structure. As shown, in some forms, the headgear structure 3350 is a continuous sleeve structure.

For example, the first layer 3352 is provided in a continuous sleeve structure and does not have any sharp or uneven edges, seams and/or joints. As shown in the figures, in some forms, the first layer 3351 only forms the sleeve 3351. The first layer 3352 may be a woven or a knitted structure. In some forms, the first layer 3352 may be formed by at least one of a narrow needle loom, a jacquard loom, and a double needle warp knit. In other forms, the first layer 3352 may be formed using any other suitable method or means known to one skilled in the art.

In some forms, the thermoformable layer 3354 is provided in a continuous sleeve structure. The thermoformable layer 3354 may not have any sharp or uneven edges, seams and/or joints. In some forms, the thermoformable layer 3354 may be positioned so that it extends over the entire perimeter defined by an inner surface of the first layer 3352. The thermoformable layer 3354 may be formed by at least one of a narrow needle loom, a jacquard loom, and a double needle warp knit, however it may be formed using any other suitable method or means known to one skilled in the art.

In some forms, the headgear structure 3350 may be a continuous sleeve-like structure. For example, the first layer 3352 and the thermoformable layer 3354 may be integrally formed in a continuous sleeve structure, e.g. a woven or knitted structure as will be described in more detail further below.

In some forms, the thermoformable layer 3354 may be heated to a predetermined temperature at which the thermoformable material will deform into a desired shape created by a shaping component, e.g. a mandrel or a mould which has a desired or predefined shape. Once the deformed thermoformable material cools, the thermoformable layer 3354 may harden to at least partially retain or hold the desired shape. This can help create the predefined shape of the headgear structure 3350. These aspects of the present technology will be discussed in further detail below.

In some forms, the thermoformable layer 3354 may be attached or joined to at least one of the other layers of the headgear structure 3350, e.g. the first layer 3352. These layers 3352, 3354 may be attached together before a thermoforming process is performed, e.g. heating or thermo-processing process as described elsewhere herein.

For example, in some forms, one of the thermoformable layer 3354 and the first layer 3352 may be coated, laminated, and/or adhered to the other one of the thermoformable layer 3354 and the first layer 3352. Adhesives such as spray glue, hotmelt, hotmelt powder, reactive hotmelt (i.e. moisture curing adhesive), and/or any co-polyester, co-polyamide adhesive may be used to attach the layers together. This may help hold the two layers 3352, 3354 in position relative to one another during a thermoforming process.

In another form, the thermoformable layer 3354 and the first layer 3352 may be integrally formed. For example, a double knit structure may be formed, e.g. a double knit fabric. The double knit structure may be formed as a continuous sleeve, and therefore may provide the headgear structure 3350 as described herein. The double knit structure comprises a technical face and a technical back. The technical face may be formed by a first knit layer, e.g. the first layer 3352, and the technical back may be formed by a second knit layer, e.g. the thermoformable layer 3354, wherein the first knit layer and the second knit layer may be knit together in the same process (i.e. they are integrally formed).

However, the thermoformable layer 3354 and one of the other layers of the headgear structure 3350, e.g. the first layer 3352, may be attached or formed together during a thermoforming process.

In some forms, the thermoformable layer 3354 and at least one of the other layers are heat bonded or thermoformed together when the thermoformable layer 3354 is heated to a predetermined temperature. The thermoformable material melts at the predetermined temperature and bonds or fuses with surrounding material, e.g. the material of at least one of the other layers of the headgear structure 3350, e.g. the first layer 3352, and/or the material of surrounding portions of the thermoformable layer 3354 (e.g. non-thermoformable material which forms part of the thermoformable layer 3354 or material which has a higher melting point than the thermoformable material comprising part of the thermoformable layer 3354). Once the thermoformable material cools, the thermoformable layer 3354 and the at least one other layer of the headgear structure 3350 are joined together. This may cause the at least one other layer of the headgear structure 3350 to correspond, conform or contour to the shape of the thermoformable layer 3354. For example, the thermoformable layer 3354 is thermoformed to the first layer 3352 to join these two layers 3352, 3354 together and provide the headgear structure 3350 with a predefined shape.

The thermoformable layer 3354 may have a lower melting point than at least one of the other layers of the headgear structure 3350. For example, the thermoformable layer 3354 may have a lower melting point than the first layer 3352. This can allow the thermoformable layer 3354 to deform into the desired shape and bond or fuse with the first layer 3352 without the first layer 3352 deforming which may affect the handle of the fabric.

FIG. 6C illustrates the first layer 3352 positioned to cover the thermoformable layer 3354. However, they do not yet have a predefined shape, i.e. they have not yet been thermo-processed.

In some forms, the thermoformable layer 3354 and the first layer 3352 may be positioned on a shaping component before being thermo-processed. For example, as illustrated in FIG. 6D, the thermoformable layer 3354 and the first layer 3352 may be positioned on a mandrel 3360. In other forms, the shaping component may be a mould or any other suitable component configured to form the headgear structure 3350 or part thereof with a predefined shape.

In some forms, the shaping component may be heated to a predetermined temperature. For example, as illustrated in FIG. 6E, the mandrel 3360 may be heated to a predetermined temperature at which the thermoformable layer 3354 is thermoformed to the first layer 3352 and takes the shape of the mandrel 3360.

In some forms, the headgear structure 3350 may then be removed from the shaping component and hold or retain its shape once removed. For example, the headgear structure 3350 may be removed from the mandrel 3360 and the headgear structure 3350 which has a predefined shape is formed, as is perhaps best illustrated in FIG. 6A.

The headgear structure 3350 may be configured to reduce, limit (or minimize) and/or prevent the creation of creases or wrinkles on the headgear structure 3350 when forming it into a 3D shape, e.g. during the thermo-processing. For instance, this may be achieved by forming a headgear structure 3350 that is stretchable in one or more directions, at least before thermo-processing.

In some forms, the headgear structure 3350 may be substantially elastic. As described above, the elasticity of the first layer 3352 and the thermoformable layer 3354 may provide this elasticity.

The elasticity of the headgear structure 3350 may allow at least a portion of it to stretch when on the shaping component, e.g. in a mould or over a mandrel 3360. When the headgear structure 3350 is thermo-processed, e.g. the thermoformable layer is thermo-bonded to the first layer 3352, the stretched portion(s) may maintain a stretched condition. This may reduce, limit (or minimize) and/or prevent the creation of creases or wrinkles when forming the headgear structure 3350 into a 3D shape.

The headgear structure 3350 may have dimensions that are smaller than the dimensions of the shaping component, e.g. the dimensions of a mould or a mandrel. For example, the headgear structure 3350 may be narrow with respect to the mandrel 3360. The headgear structure 3350 may be formed as a narrow hollow structure, e.g. a sleeve. This configuration may permit stretching of the headgear structure 3350 as described above.

The elasticity of the headgear structure 3350 may have one or more other benefits. For instance, examples of the benefits, include:
allowing the mandrel 3360 and headgear structure 3350 to be more easily separated after thermoforming;
allowing differently sized shaping components, e.g. a thicker mandrel, or a thinner mandrel, to be used to shape and/or dimension the headgear structure 3350; and
allowing shaping components which have regions with varying sizes, e.g. mandrels with varying thicknesses, to be used to shape and/or dimension the headgear structure 3350.

In some forms, the headgear structure 3350 can flex, bend, and/or be compressed from its predefined shape, e.g. when handling the headgear structure 3350. For example, the headgear structure 3350 may be substantially flexible, i.e. it is not rigid. This may facilitate fitting or removal of the headgear structure 3350 to a patient.

The headgear structure 3350 may be configured to return to its predefined shape. For example, the headgear structure 3350 may be substantially resilient or be configured to act resiliently. This can allow it to flex, bend, and/or be compressed as described above, but return to its predefined shape. The thermoformable layer 3354, when thermoformed to the inner surface of the sleeve 3351, may configure the headgear structure 3350 to be substantially resilient or to act resiliently such that the headgear structure returns to the predefined shape when flexed, bent, and/or compressed from the predefined shape. This may assist with useability in contactless or remote fitting of patients. For example, the patient interface 3000 and its components can be delivered to the patient, e.g. the headgear structure may be flexed, bent and/or compresses (i.e. folded) to reduce the size of the packaging required. When a person e.g. patient removes the patient interface 3000 and components from the packaging, the headgear structure 3350 returns to its predefined shape. The predefined shape may be indicative of how the patient interface 3000 should be worn on the patient, thereby facilitating ease of fitting and use.

Fixing the thermoformable layer 3354 and the first layer 3354 may provide a stiffened or rigidised headgear structure 3350. By stiffening or rigidising the headgear structure 3350 in this way, the headgear structure 3350 is provided with a predefined shape. Furthermore, fixing the thermoformable layer 3354 and the first layer 3352 may provide a headgear structure 3350 that is rigid or stiffened enough to hold it in a predefined shape, but that is still flexible enough to flex, bend and/or be compressed in one or more directions and return to or otherwise adopt the predefined shape again once any compression, flexing or bending forces are removed.

The headgear structure 3350 with a predefined shape may be substantially lightweight relative to existing headgear structures formed with a predefined shape. For example, the sleeve-like or hollow nature of the headgear structure 3350 may make it lighter. In addition to this, or alternatively, the materials used to form the headgear structure may make it lighter. The materials that can be used should become clearer from the discussion herein.

It should be apparent to one skilled in the art that the present technology may provide a number of benefits, these can include a headgear structure 3350 that is one or more of the following:
formed in a predefined shape which contours parts of the patient's face and/or head in use;
substantially flexible;
substantially resilient;
has fewer or no edges, seams or joints around a perimeter or circumference defined by the headgear structure; and/or
substantially lightweight.

These benefits may improve patient comfort and fit, and increase compliance with therapy.

However, in other forms, the headgear structure 3350 may be configured to reduce, limit (or minimize) and/or prevent flexing, bending, and/or compression in one or more regions. For example, the headgear structure 3350 may be substantially rigid or rigidised in one or more regions. This may allow the headgear structure 3350 to better hold the predefined shape. The rigidised headgear structure 3350 may be partially flexible, in that it may allow for some bending, flexing and/or compression in one or more regions, however, the rigidised headgear structure 3350 is rigid relative to the substantially flexible headgear structure 3350 described above, i.e. the rigidised headgear structure 3350 is less flexible or deformable.

The rigidiser, e.g. the thermoformed thermoformable layer 3354, may be stiffer, more rigid, or harder with respect to at least one of other layers, e.g. the first layer 3352. Therefore, in addition to helping shape the headgear structure 3350, the thermoformed thermoformable layer 3352 may help stiffen, rigidise or harden the headgear structure 3350. This may help reduce, limit (or minimize) and/or prevent flexing, bending, and/or compression of the headgear structure 3350 as described above.

Referring now to FIGS. 7A to 7E which illustrate an embodiment of the headgear structure 3350 which comprises a rigidiser component 3370.

FIG. 7A illustrates a headgear structure 3350 that does not yet have a predefined shape.

In some forms, the headgear structure 3350 may have a sleeve 3351 and a thermoformable layer 3354 as described above.

FIG. 7B illustrates a headgear structure 3350 formed with a predefined shape. The illustrated headgear structure 3350 has a substantially curved shape, however, the headgear structure 3350 may have any other suitable predefined shape.

In some forms, as shown in FIGS. 6A to 6C, 7A and 7C, the headgear structure 3350 may be configured to define a cavity 3356. The cavity 3356 may extend along at least a portion of the length of the headgear structure 3350, preferably, along the entire length, as shown.

In some forms, the thermoformable layer 3354 may be thermoformed to the inner surface of the sleeve 3351 to form the inner surface 3382 of the headgear structure 3350. The inner surface 3382 surrounds the cavity 3356.

Therefore, in some forms, when the thermoformable layer 3354 is thermoformed to the inner surface of the sleeve 3351 and gives it a predefined shape, portions of the inner surface 3382 of the headgear structure 3350 are spaced apart such that the cavity 3356 is held open. In other words, a gap is formed between opposing portions of the inner surface 3382.

The headgear structure 3350 may be configured to hold at least one rigidiser component 3370. For example, as described above, the headgear structure 3350 may be configured to be hollow, e.g. it is a hollow headgear structure. In other words, it is unfilled, or defines a space or gap inside. The inner surface 3382 of the headgear structure 3350 may surround the hollow.

In some forms, the rigidiser component 3370 can be positioned (e.g. inserted) in the cavity 3356, as illustrated in FIGS. 7C and 7D.

FIG. 7E illustrates the rigidiser component 3370. In some forms, the rigidiser component 3370 may have a predefined shape. The shape of the rigidiser component 3370 may contour or correspond to the predefined shape of the headgear structure 3350. In other words, the rigidiser component 3370 may be formed with a similar or the same shape as the headgear structure 3350 (with a predefined shape). The illustrated rigidiser component 3370 has a curved shape, e.g. along its length, however, it may have any other suitable predefined shape, which may extend in one or more other directions. The rigidiser component 3370 may be substantially flat or planar. For example, the rigidiser component 3370 may be substantially thin, e.g. a thickness of about 1 mm to about 1.5 mm.

The rigidiser component(s) 3370 may help the headgear structure 3350 (with a predefined shape) hold its shape. The rigidiser component(s) 3370 may be used to rigidise and support portions of the positioning and stabilising structure 3300. The rigidiser component(s) 3370 may be provided in sections of the headgear structure 3350 which may help support and rigidise the sections.

In some forms, the rigidiser component 3370 and the headgear structure 3350 may be removably attached to each other. That is, the rigidiser component 3370 can be inserted into and removed from the headgear structure 3350 as required. This may facilitate cleaning and/or replacement of components of the patient interface 3000. Therefore, the headgear structure 3350 may comprise at least one opening which connects the cavity 3356 to outside the headgear structure 3350. This may allow insertion and removal of the rigidiser component 3370 from the cavity.

In other forms which are not illustrated, the rigidiser component 3370 and the headgear structure 3350 may be permanently attached or formed together. For example, in some forms, the rigidiser component 3370 may be inserted into the cavity 3356 before the thermo-processing. The headgear structure 3350 and rigidiser component 3370 may then positioning in or on a heat press or mould. The sleeve 3351 may then be heat pressed or moulded together with the rigidiser component 3370 so that the thermoformable layer 3354 is bonded to at least one of the rigidiser component 3370 and one of the other layers, e.g. the first layer 3352. The thermoformable layer 3354 may have a lower melting point than the rigidiser component 3370. This arrangement may allow the thermoformable layer 3354 to bond or fuse with the rigidiser component 3370 without the rigidiser component deforming during the thermo-processing which could impact the integrity and shape of the rigidiser component 3370.

In other forms (which are not illustrated), a rigidiser component may be formed by adding a material into the cavity 3356 before or during the application of heat to a headgear structure 3350 that has the sleeve 3351 and the thermoformable layer 3354. For example, the material may be a soft material, e.g. a fluid material, which can set, cool or cure to harden, such as a molten material. In some forms, the temperature of the soft material added into the cavity 3356 may apply the heat at the temperature required to deform the thermoformable layer 3354 in the mould such that it thermoforms to the sleeve 3351. Alternatively, the heat may be applied during a thermo-processing step. Once the material cools, the headgear structure 3350 can be removed from the mould or heat press and it has a predefined shape.

In some forms, the thermoformable material comprises a thermoplastic element, e.g. a thermo-fusible yarn 3354a and reference herein will be made as such.

The thermoformable material may comprise at least one of polypropylene, polyethylene, thermoplastic polyurethane, thermoplastic elastomer, amongst other materials that would be known to one skilled in the art. Depending on the material used for the thermoformable material, the thermoformable layer 3354 may further comprise at least one support element e.g. a support yarn 3354b, that supports the at least one thermoformable material, and reference herein will be made as such. The thermoformable material may have a lower softening temperature than the support yarn 3354b. Therefore, the thermo-fusible yarn 3354a may have a lower melting point than the support yarn 3354b. For example, the support yarn 3354b may be nylon or polyester, e.g. a nylon yarn or polyester yarn, while the thermoformable material may be polypropylene, polyethylene, thermoplastic polyurethane or thermoplastic elastomer.

As described above, the headgear structure 3350 may comprise a double knit structure. The technical face may be softer than the technical back. The technical face may form an outer surface 3380 of the headgear structure 3350, which contacts the patient's skin in use.

The technical back may be stiffer than the technical face. The technical back may form an inner surface 3382 of the headgear structure 3350, which does not contact the patient's skin in use. The thermo-fusible yarn 3354a may be incorporated into the double knit structure at the technical back. This may make the double knit structure stiffer on the inner surface 3382. As a result, the integrity of the technical face, or outer surface 3380, may be maintained. For instance, the soft feel of the technical face, or outer surface 3380 may be maintained.

At least one of the layers of the headgear structure 3350 may be configured to reduce, limit (or minimize) and/or prevent adhesives and/or a thermoformable material from flowing into at least one other layer of the headgear structure 3350, e.g the first layer 3352 which forms the outer layer of the headgear structure 3350, and cooling, setting or curing there. For example, the technical back, or inner surface 3382 may be substantially resistant to penetration by adhesive, e.g. it may be substantially impermeable. This may ensure that the headgear structure 3350 retains a comparatively soft feel or touch, and is less likely to be uncomfortable or perceived as such.

Referring to FIGS. 8A to 8D, the thermoformable layer 3354 may be formed using various techniques.

The thermo-fusible yarn 3354a may be knitted or woven with the support yarn 3354b yarn during manufacture of a fabric. The thermo-fusible yarn 3354a can replace one of the warp and/or weft yarns in the fabric. Alternatively, the thermo-fusible yarn 3354a can be knitted or woven throughout the fabric or in one or more localized patterns in the fabric. The warp thread and weft thread may have different melting temperatures.

The thermo-fusible yarn 3354a can be covered or twisted with a support yarn 3354b. For example, a thermo-fusible yarn may be combined with a core yarn to create a dual-strand yarn, e.g. the yarns are twisted with each other in the same or opposite twisting direction. A suitable spun core yarn includes an elastic yarn which may have a non-elastic short fibre which extends in the direction of the elastic yarn so that the non-elastic short fibre assembly encloses the circumference of the elastic yarn as a core.

The density and/or thickness of the thermo-fusible yarn 3354a may be varied. In one example, a nylon yarn may have a density of about 15 Denier to about 280 Denier. In another example, a polyester yarn may have a density of about 20Denier to about 150 Denier.

The density and/or thickness of the support yarn 3354b may be varied. A support yarn made of nylon, polyester, or polyurethane co-polymer can have a density of about 15 Denier to about 210 Denier.

The yarn density may be important for the fabric or layers hand-feel, the thickness of the fabric or layers, and/or may help make regions of fabric or layers softer or stiffer.

For example, in the embodiment of FIG. 8A, the thermo-fusible yarn 3354a may be woven with the support yarn 3354b. One of the warp thread and the weft thread may comprise the support yarn 3354b (e.g. a polyester or nylon yarn), and the other one of the warp thread and the weft thread may comprise a thermo-fusible yarn 3354a. In the embodiment of FIG. 8A, the warp thread comprises the support yarn 3354b, and the weft thread comprises the thermo-fusible yarn 3354a.

For example, in the embodiment of FIG. 8B, the thermo-fusible yarn 3354a may be warped with the support yarn 3354b.

For example, in the embodiment of FIGS. 8C and 8D, the thermo-fusible yarn 3354a may be braided with the support yarn 3354b.

As described above, the thermoformable layer 3354 may be provided as a continuous sleeve structure. The support yarn 3354b and the thermo-fusible yarn 3354a may be woven, warped or braided into the thermoformable layer 3354.

In some forms, the thermoformable layer 3354 may be configured to stretch in a first direction, e.g. along its length, and restrict stretching in a second direction, e.g. along its width. Therefore, the first and second direction may be different from each other. For example, the thermo-fusible yarn 3354a may be positioned on the thermoformable layer 3354 so that the thermo-fusible yarn 3354a extends across at least a portion of the width of the thermoformable layer 3354, preferably across the entire width of the thermoformable layer 3354. As described above, at least one of the other layers, particularly the first layer 3352, may be configured to stretch in at least one direction, e.g. at least along its length, or along its length and width (i.e. it can stretch in a plurality of directions). This may permit the headgear structure 3350 to stretch along its length but restrict stretching along the width of the headgear structure 3350.

This configuration may help reduce, limit (or minimize) and/or prevent movement of the positioning and stabilising structure 3300 in directions which can affect the seal that the seal-forming structure 3100 has with the patient's face, e.g. movement in a substantially superior or inferior direction relative to the patient's head and/or face.

This configuration may allow movement of the positioning and stabilising structure 3300 which facilitates fitting the patient interface 3000 to or removal from the patient's head, e.g. movement in a substantially lateral direction relative to the patient's head and/or face.

In other forms (not shown), the amount of thermoformable material may be varied to create a headgear structure 3350 with varying stiffnesses or rigidity. For example, the amount of thermo-fusible yarn 3354a included in the thermoformable layer 3354 may be varied according to the stiffness or rigidity desired, and/or the shape desired. For example, a headgear structure 3350 with a substantially low rigidity may incorporate about 5% to about 25% thermoformable material in the thermoformable layer 3354, e.g. about 10%. Such a configuration would be more flexible, and therefore more easily deformable from its predefined shape when compared to a headgear structure 3350 with a substantially higher rigidity.

In other forms (not shown), a first region of the headgear structure may be configured to be harder, stiffer or more rigid than a second region of the headgear structure. For example, a section (e.g. a first section) of the thermoformable layer 3354, which may be located in the first region of the headgear structure 3350 may incorporate a higher amount, e.g. percentage, of thermo-fusible yarn with respect to another section (e.g. a second section) of the thermoformable layer 3354 located in the second region of the headgear structure 3350. This provides a harder first section with respect to the second section. In yet other forms, a headgear structure 3350 with sections with varying hardness, stiffness or rigidity may be formed by providing one or more rigidiser components, e.g. rigidiser component 3370, as described herein, to one or more sections of the headgear structure 3350.

In other forms, at least one of the other layers of the headgear structure 3350, e.g. the first layer 3352 described above, may be thermoformable and comprise a thermoformable material. For example, the first layer 3352 above may include sections which incorporate at least one thermoplastic element, e.g. a thermo-fusible yarn.

In other forms not shown, at least one of the layers of the headgear structure 3350 may be less permeable than at least one of the other layers. For example, at least one of the layers of the headgear tube 3350 may be substantially impermeable, e.g. gas impermeable, to provide a flow path. This may allow a headgear structure to be formed that can be used as or part of a headgear tube for a patient interface comprising headgear tubing (i.e. a conduit headgear mask system).

### 4.3.3.2 Second Embodiment of Headgear Structure

Referring now to FIGS. 9A to 9E which illustrate a second embodiment of a headgear structure 3350A according to an aspect of the present technology.

In some forms, the headgear structure 3350A comprises a plurality of layers of material.

In some forms, the headgear structure 3350A comprises a first layer 3352A which is similar to the first layer 3352 described above, e.g. it is made from the same or similar material(s). Therefore, first layer 3352A may comprise the features and benefits of the first layer 3352 as described above. Therefore, in some forms, the first layer 3352A has an outer surface which forms the outer surface 3380 of the headgear structure 3350 which contacts the patient's face and/or head in use.

In some forms, at least one of the layers may be less permeable than at least one of the other layers. For example, as is perhaps best illustrated in FIG. 9B, the headgear structure 3350A comprises a second layer 3354A that may be less permeable than the first layer 3352A. The second layer 3355 may be substantially impermeable, e.g. gas and/or liquid impermeable.

The headgear structure 3350A may stretch in one or more directions. For example, the first layer 3352A may be elastic, as described herein. In some forms, the second layer 3355 may be substantially elastic. For example, the second layer 3355 may comprise an elastic material. This may allow the second layer 3355 to stretch in at least one direction, e.g. along its length.

In some forms, the second layer 3355 may be positioned against at least a portion of one of the other layers. In some forms, the second layer 3355 is positioned radially inwards from the first layer 3352A. For example, the second layer 3355 may be positioned with respect to the first layer 3352A so that it extends over at least a portion of an inner surface of the first layer 3352A. As shown in FIGS. 9A to 9E, the second layer 3355 is positioned to extend over the entire inner surface of the first layer 3352A, as shown in FIGS. 9A to 9E. However, in other forms, as shown in FIG. 6F, the second layer 3355 may be positioned to extend over a portion of the inner surface of the first layer 3352A. Therefore, the second layer 3355 may form an inner layer and the first layer 3352A may form an outer layer which may contact the patient's face and/or head in use.

In some forms, the second layer 3355 may be thinner than the first layer 3352A, as shown in FIG. 9B. That is, the dimensions between an inner surface and an outer surface of the second layer 3355 are smaller or less than the dimensions between an inner surface and an outer surface of the first layer 3352A. However, in other forms not shown, the thermoformable layer 3354 and the first layer 3352 may have substantially the same thickness, or the second layer 3355 may be thicker than the first layer 3352A.

In some forms, the second layer 3355 and the first layer 3352A may be attached together. For example, the second layer 3355 and the first layer 3352A may be welded, laminated, thermo-bonded, stitched or adhered (e.g. using adhesives) together.

The headgear structure 3350A may comprise a plurality of portions or sections which may be separately formed and joined together. For example, the portions or sections may be welded, laminated, thermo-bonded, stitched or adhered (e.g. using adhesives) together.

For example, the headgear structure 3350A may comprise a first portion, e.g. first half 3353A, and a second portion, e.g. second half 3353B, which are joined. The first half 3353A and the second half 3353B may each comprise lateral end regions 3357A which extend along the length of the first half 3353A and the second half 3353B. The first half 3353A and the second half 3353B may be attached to each other at the respective lateral end regions 3357A. For example, the lateral end regions 3357A may be attached together by thermo-bonding. Other means of joining may be used, as described above, e.g. welding, laminating, etc. For example, in some forms, the lateral end regions 3357A are attached to each other using an adhesive.

As illustrated, the first half 3353A provides a patient facing side of the headgear structure 3350A, and the second half 3353B provides a non-patient facing side of the headgear structure 3350A.

As shown in FIG. 9F, in some forms, part of the second layer 3355 on each of the first half 3353A and the second half 3353 may be positioned such that it extends over a portion of the inner surface of part of the first layer 3352A on each of the first half 3353A and the second half 3353B. In some forms, the parts of the second layer 3355 may not extend to the periphery or edge of the lateral end regions 3357A. In some forms, these parts of the second layer 3355 may be positioned so that they surround the cavity 3356A only, as shown. In other forms not shown, the second layer 3355 may be continuous (i.e. not formed as part of two halves which are joined), and the first layer 3352A may comprise a first part and a second part which are joined at the lateral end regions thereof, wherein the second layer 3355 is positioned to cover a portion of an inner surface of first part and a portion of an inner surface of the second part.

In some forms, which are not shown, a joint between the inner surfaces of the first part and the second part of the first layer 3352A may be covered by the second layer 3355, or parts of the second layer 3355. The second layer 3355, or parts of it, may be positioned to cover the joint. For example, the continuous second layer 3355 described above may cover the joint. This arrangement may limit or prevent any core material flowing into the lateral edge regions 3357A.

As is perhaps best illustrated in FIGS. 9C and 9D, the headgear structure 3350A may comprise a sleeve 3351A. The sleeve 3351A may comprise a cavity 3356A. At least one of the layers of the headgear structure 3350A may be arranged to define the cavity 3356A. For example, the second layer 3355 may be arranged to define the cavity 3356A in the sleeve 3351A. As illustrated, in some forms, the two halves 3353A, 3353B may be joined to form the sleeve 3351A.

In some forms, the sleeve 3351A may comprises an outer surface and an inner surface. The outer surface of the sleeve 3351A may form the outer surface 3382 of the headgear structure which contacts the patient's face and/or head in use.

In some forms, the sleeve 3351A may comprise an outer layer which is formed by the first layer 3352A. This layer 3352A provides the outer surface of the sleeve 3351A.

In some forms, the sleeve 3351A may comprise an inner layer which is formed by the second layer 3355. This layer 3355 provides the inner surface of the sleeve 3351A.

As illustrated, the sleeve 3351A may be formed by the half 3353A and the second half 3353B when they are attached together as described above. However, in other forms not shown, a sleeve may be formed in a continuous sleeve, wherein the continuous sleeve does not have edges, seams or joints. This sleeve may have a similar configuration to the sleeve 3351 described earlier.

In some forms, the headgear structure 3350A comprises a core 3358A. The core 3358A may be formed inside the cavity 3356A. As illustrated, the core 3358A may be positioned inside the sleeve 3351A between portions of the second layer 3355.

The core 3358A may be configured to provide a desired shape to the headgear structure 3350A. The core 3358A is positioned inside the sleeve 3351A to provide a pre-defined shape to the sleeve 3351A.

The sleeve 3351A and the core 3358A may be shaped using a shaping component which has a predefined shape. For example, the sleeve 3351A and the core 3358A may be moulded into a desired shape, e.g. using a mould 3360A which has a predefined shape, as shown in FIG. 9E.

The core 3358A may comprise a core material. In some forms, the core 3358A may be formed inside the sleeve 3351A from a soft material positioned inside the sleeve 3351A which hardens to form the core 3358A. The core material includes a soft material, e.g. a fluid material, which can cool, cure or set to harden. For example, the core material 3358A may comprise at least one of a molten thermoplastic material, a liquid polymer, and a polyurethane foam resin.

In some forms, the core 3358A may stretch in one or more direction. For instance, the core 3358A may be substantially elastic. The core material may comprise an elastic material.

The core 3358A may be stiffer, more rigid, or harder with respect to the sleeve 3351A. Therefore, the core may help stiffen, rigidise or harden the headgear structure 3350A.

In some forms, the fluid material may be injected into the cavity 3356A. The sleeve 3351A containing injected fluid material may be placed in a mould 3360A and moulded into a desired shape. Once the injected fluid material cools, cures or sets, the headgear structure 3350A can be removed from the mould 3360A. The headgear structure 3350A formed can hold its shape.

In other forms (not illustrated), the core material includes a thermoset material, e.g. resin powder. The thermoset material may be added into the cavity 3356A and fused or thermoset by addition of at least one of heat applied and chemicals added into the cavity 3356A. The sleeve 3351A and added thermoset material may be moulded to form a headgear structure that can hold its shape.

The headgear structure 3350A may have a patient contacting portion 3359 which contacts the patient's face and/or head in use. The first portion 3353A may provide the patient contacting portion 3359.

The cured or set core 3358A may be shaped so that the lateral end regions 3357A are positioned away from the patient contacting portion 3359. The patient contacting portion 3359 may be positioned on the patient facing side of the headgear structure. As mentioned above, the headgear structure 3350A may also have a non-patient facing side.

In some forms, at least one of the patient facing side and the non-patient facing side of the headgear structure 3350A is substantially convex-shaped, when viewed in cross-section from a side in a direction substantially parallel to a longitudinal axis of the headgear structure (shown in FIG. 9B). That is, the first half 3353A and/or the second half 3353B may be substantially convex-shaped. For example, the core 3358A may have a substantially oval-shaped cross-section, or a substantially lens-shaped cross-section as shown. The sleeve 3351A may contour or correspond to the shape of the core 3358A. However, the core 3358A may be formed with any other suitable shaped cross-section, e.g. any shaped cross-section that helps position the lateral end regions 3357A away from the patient contacting portion 3359. This arrangement may limit or prevent irritation and/or the formation of marks on the patient's face and/or head when using the patient interface 3000, and may improve patient comfort and fit. This may lead to increased therapy compliance.

As illustrated, in some forms, the first half 3353A and the second half 3353B each comprise at least one layer, preferably a plurality of layers. For example, each of the first half 3353A and the second half 3353B comprise part of the first layer 3352A and part the second layer 3355 of the sleeve 3351A. The core 3358A may be positioned between parts of the second layer 3355 provided by each of the first half 3353A and the second half 3353B.

In some forms, at least one of the layers of the headgear structure 3350A is configured to line at least a portion of the cavity 3356A, preferably the entire cavity 3356A. For example, the second layer 3355 lines the entire perimeter defined by the cavity 3356A.

In some forms, at least one of the layers may be configured to reduce, limit (or minimize) and/or prevent the core material and/or adhesives (e.g. used to join the first portion 3353A and second portion 3353B) from flowing into at least one other layer of the headgear structure 3350A, particularly the first layer 3352A, and cooling, setting or curing there. In some forms, the second layer 3355, i.e the inner layer of the sleeve 3351A, limits or prevents the soft core material from flowing into the first layer 3352A, i.e. the outer layer of the sleeve 3351A, before soft core material hardens to form the core 3358A. In some forms, the second layer 3355 limits or prevents the adhesive from flowing into the first layer 3352A, before the adhesive sets, hardens or cures. For example, as described above, the second layer 3355 may be less permeable than the first layer 3352A, e.g. the second layer 3355 may be substantially impermeable. This may help the headgear structure 3350A retain a comparatively soft feel or touch, and is less likely to be uncomfortable or perceived as such.

However, it is to be appreciated that in some forms the core 3358A may at least be partially integrated into a portion of the sleeve 3351A. For example, some of the soft core material may flow into at least one of the other layers of the headgear structure 3350A when providing the core material into the cavity 3356A and cure there, e.g. the core material may be melted and integrate into the fibres forming the second layer 3355, and subsequently harden on curing to form a bond between the rigidiser and the material. However, as described herein, the at least one other layer, e.g. the second layer 3355, is configured to reduce, limit (or minimize) and/or prevent the core material from flow through into the first layer 3352A because it is less permeable than the first layer 3352A.

### 4.3.4 Vent

In one form, the patient interface 3000 includes a vent 3400 constructed and arranged to allow for the washout of exhaled gases, e.g. carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO2 by the patient while maintaining the therapeutic pressure in the plenum chamber in use.

One form of vent 3400 in accordance with the present technology comprises a plurality of holes, for example, about 20 to about 80 holes, or about 40 to about 60 holes, or about 45 to about 55 holes.

The vent 3400 may be located in the plenum chamber 3200. Alternatively, the vent 3400 is located in a decoupling structure, e.g., a swivel.

### 4.3.5 Decoupling Structure(s)

In one form the patient interface 3000 includes at least one decoupling structure, for example, a swivel or a ball and socket.

### 4.3.6 Connection Port

Connection port 3600 allows for connection to the air circuit 4170.

### 4.3.7 Forehead Support

In one form, the patient interface 3000 includes a forehead support 3700.

### 4.3.8 Anti-Asphyxia Valve

In one form, the patient interface 3000 includes an anti-asphyxia valve.

### 4.3.9 Ports

In one form of the present technology, a patient interface 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

### 4.4 Method of Manufacturing Headgear Structure

Forms of the technology provide a method of manufacturing a headgear structure 3350, 3350A with a predefined shape.

In certain forms, the method may comprise the following steps, occurring in any order:
(a) forming at least one of the headgear structure 3350 and the headgear structure 3350A; and
(b) shaping the respective headgear structure 3350, 3350A.

### 4.4.1 Method of Manufacturing First Embodiment of Headgear Structure

Forms of the technology provide a method of manufacturing the headgear structure 3350.

In certain forms, step (a) comprises forming the thermoformable layer 3354, e.g. from a thermo-fusible yarn 3354a and a support yarn 3354b.

In certain forms, step (a) comprises forming the at least one other layer, e.g. the first layer 3352.

In certain forms, step (a) comprises positioning the thermoformable layer 3354 against the at least one other layer, e.g. the first layer 3352.

In certain forms, step (b) comprises positioning the at least one other layer and the thermoformable layer 3354 on a shaping component, e.g. positioning the first layer 3352 and the thermoformable layer 3354 over the mandrel 3360.

In certain forms, step (b) comprises applying heat to the thermoformable layer 3354 so that the thermoformable layer 3354 is thermo-bonded to the at least one other layer, e.g. to the first layer 3352. For example, this may be achieved by heating the shaping component, e.g. the mandrel 3360.

The method may further comprise the step of removing the thermo-bonded thermoformable layer 3354 and the at least one other layer from the shaping component. For example, the thermo-bonded thermoformable layer 3354 and first layer 3352 can be removed from the mandrel 3360.

### 4.4.2 Method of Manufacturing Second Embodiment of Headgear Structure

Forms of the technology provide a method of manufacturing the headgear structure 3350A.

In certain forms, step (a) comprises forming the first layer 3352A.

In certain forms, step (a) comprises forming the second layer 3354A.

In certain forms, step (a) comprises arranging the second layer 3354A to form the cavity 3356A for the sleeve 3351A.

In certain forms, step (a) comprises positioning the second layer 3354A against the first layer 3352A. For example, this may include attaching the second layer 3354A to the first layer 3352A.

In certain forms, step (a) comprises forming the first portion, e.g. forming the first half 3353A comprising part of the first layer 3352A and part of the second layer 3354A.

In certain forms, step (a) comprises forming the second portion, e.g. forming the second half 3353B comprising part of the first layer 3352A and part of the second layer 3354A.

In certain forms, step (a) comprises attaching the first portion, e.g. the first half 3353A, and the second portion, e.g. the second half 3353B, together. For example, this step may include attaching the lateral end regions 3357A of the first half 3353A and the second half 3353A together.

In certain forms, step (a) comprises forming the headgear structure 3350A as a sleeve 3351A which provides the cavity 3356A.

In certain forms, step (a) comprises forming the headgear structure 3350A comprising the core 3358A. For example, this step may include adding, positioning, or providing the core material into the cavity 3356A.

In certain forms, step (b) comprises heating the core material to melt it.

In certain forms, step (b) comprises allowing the melted, molten or soft core material to cool to harden.

In certain forms, step (b) comprises allowing the core material to set or cure to harden.

In certain forms, step (b) comprises allowing the core material to at least partially integrate into the second layer 3355. For instance, this step may include melting the core material into the second layer 3355.

In certain forms, step (b) comprises positioning the sleeve 3351A and the core 3358A in the mould 3360A.

In certain forms, step (b) comprises moulding the sleeve 3351A and the core 3358A.

The method may further comprise the step of removing the moulded sleeve 3351A and core 3358A from the mould 3360A.

### 4.5 RPT Device

An RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document.

### 4.5.1 RPT Device Algorithms

As mentioned above, in some forms of the present technology, the central controller 4230 may be configured to implement one or more algorithms 4300 expressed as computer programs stored in a non-transitory computer readable storage medium, such as memory 4260. The algorithms 4300 are generally grouped into groups referred to as modules.

In other forms of the present technology, some portion or all of the algorithms 4300 may be implemented by a controller of an external device such as the local external device 4288 or the remote external device 4286. In such forms, data representing the input signals and/or intermediate algorithm outputs necessary for the portion of the algorithms 4300 to be executed at the external device may be communicated to the external device via the local external communication network 4284 or the remote external communication network 4282. In such forms, the portion of the algorithms 4300 to be executed at the external device may be expressed as computer programs stored in a non-transitory computer readable storage medium accessible to the controller of the external device. Such programs configure the controller of the external device to execute the portion of the algorithms 4300.

In such forms, the therapy parameters generated by the external device via the therapy engine module 4320 (if such forms part of the portion of the algorithms 4300 executed by the external device) may be communicated to the central controller 4230 to be passed to the therapy control module 4330.

### 4.6 Air Circuit

An air circuit 4170 in accordance with an aspect of the present technology is a conduit or a tube constructed and arranged to allow, in use, a flow of air to travel between two components such as RPT device 4000 and the patient interface 3000 or 3800.

In particular, the air circuit 4170 may be in fluid connection with the outlet of a pneumatic block and a patient interface. The air circuit 4170 may be referred to as an air delivery tube. In some cases there may be separate limbs of the circuit for inhalation and exhalation. In other cases a single limb is used.

In some forms, the air circuit 4170 may comprise one or more heating elements configured to heat air in the air circuit, for example to maintain or raise the temperature of the air. The heating element may be in a form of a heated wire circuit, and may comprise one or more transducers, such as temperature sensors. In one form, the heated wire circuit may be helically wound around the axis of the air circuit 4170. The heating element may be in communication with a controller such as a central controller. One example of an air circuit 4170 comprising a heated wire circuit is described in U.S. Pat. No. 8,733,349, which is incorporated herewithin in its entirety by reference.

### 4.7 Humidifier

### 4.7.1 Humidifier Overview

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240.

### 4.8 Glossary

For the purposes of the present technology disclosure, in certain forms of the present technology, one or more of the following definitions may apply. In other forms of the present technology, alternative definitions may apply.

### 4.8.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. ‘Flow rate’ is sometimes shortened to simply ‘flow’ or ‘airflow’.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar˜0.001 atm). In this specification, unless otherwise stated, pressure is given in units of cmH₂O.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

### 4.8.1.1 Materials

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240

Polycarbonate: a thermoplastic polymer of Bisphenol-A Carbonate.

### 4.8.1.2 Mechanical Properties

Resilience: Ability of a material to absorb energy when deformed elastically and to release the energy upon unloading.

Resilient: Will release substantially all of the energy when unloaded. Includes e.g. certain silicones, and thermoplastic elastomers.

Hardness: The ability of a material per se to resist deformation (e.g. described by a Young's Modulus, or an indentation hardness scale measured on a standardised sample size).
‘Soft’ materials may include silicone or thermo-plastic elastomer (TPE), and may, e.g. readily deform under finger pressure.
‘Hard’ materials may include polycarbonate, polypropylene, steel or aluminium, and may not e.g. readily deform under finger pressure.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

Floppy structure or component: A structure or component that will change shape, e.g. bend, when caused to support its own weight, within a relatively short period of time such as 1 second.

Rigid structure or component: A structure or component that will not substantially change shape when subject to the loads typically encountered in use. An example of such a use may be setting up and maintaining a patient interface in sealing relationship with an entrance to a patient's airways, e.g. at a load of approximately 20 to 30 cmH2O pressure.

As an example, an I-beam may comprise a different bending stiffness (resistance to a bending load) in a first direction in comparison to a second, orthogonal direction. In another example, a structure or component may be floppy in a first direction and rigid in a second direction.

### 4.8.2 Respiratory Cycle

Apnea: According to some definitions, an apnea is said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort, despite the airway being patent. A mixed apnea occurs when a reduction or absence of breathing effort coincides with an obstructed airway.

Hypopnea: According to some definitions, a hypopnea is taken to be a reduction in flow, but not a cessation of flow. In one form, a hypopnea may be said to have occurred when there is a reduction in flow below a threshold rate for a duration. A central hypopnea will be said to have occurred when a hypopnea is detected that is due to a reduction in breathing effort.

Hyperpnea: An increase in flow to a level higher than normal.

Upper airway obstruction (UAO): includes both partial and total upper airway obstruction. This may be associated with a state of flow limitation, in which the flow rate increases only slightly or may even decrease as the pressure difference across the upper airway increases (Starling resistor behaviour).

Ventilation (Vent): A measure of a rate of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 4.8.3 Anatomy

### 4.8.3.1 Anatomy of the Face

Ala: the external outer wall or “wing” of each nostril (plural: alar)

Auricle: The whole external visible part of the ear.

(nose) Bony framework: The bony framework of the nose comprises the nasal bones, the frontal process of the maxillae and the nasal part of the frontal bone.

(nose) Cartilaginous framework: The cartilaginous framework of the nose comprises the septal, lateral, major and minor cartilages.

Otobasion superior: The highest point of attachment of the auricle to the skin of the face.

Sagittal plane: A vertical plane that passes from anterior (front) to posterior (rear). The midsagittal plane is a sagittal plane that divides the body into right and left halves.

### 4.8.3.2 Anatomy of the Skull

Nasal bones: The nasal bones are two small oblong bones, varying in size and form in different individuals; they are placed side by side at the middle and upper part of the face, and form, by their junction, the “bridge” of the nose.

Nasion: The intersection of the frontal bone and the two nasal bones, a depressed area directly between the eyes and superior to the bridge of the nose.

Occipital bone: The occipital bone is situated at the back and lower part of the cranium. It includes an oval aperture, the foramen magnum, through which the cranial cavity communicates with the vertebral canal. The curved plate behind the foramen magnum is the squama occipitalis.

Parietal bones: The parietal bones are the bones that, when joined together, form the roof and sides of the cranium.

Zygomatic bones: The face includes two zygomatic bones, located in the upper and lateral parts of the face and forming the prominence of the cheek.

### 4.8.4 Patient Interface

Anti-asphyxia valve (AAV): The component or sub-assembly of a mask system that, by opening to atmosphere in a failsafe manner, reduces the risk of excessive CO2 rebreathing by a patient.

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Headgear: Headgear will be taken to mean a form of positioning and stabilizing structure designed for use on a head. For example the headgear may comprise a collection of one or more straps, struts, ties and stiffeners configured to locate and retain a patient interface in position on a patient's face for delivery of respiratory therapy. Some ties are formed of a soft, flexible, elastic material such as a laminated composite of foam and fabric.

Membrane: Membrane will be taken to mean a typically thin element that has, preferably, substantially no resistance to bending, but has resistance to being stretched.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Stiffener: A stiffener will be taken to mean a structural component designed to increase the bending resistance of another component in at least one direction.

Strut: A strut will be taken to be a structural component designed to increase the compression resistance of another component in at least one direction.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

Tie (noun): A structure designed to resist tension.

Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 4.8.5 Shape of Structures

Products in accordance with the present technology may comprise one or more three-dimensional mechanical structures, for example a mask cushion or an impeller. The three-dimensional structures may be bounded by two-dimensional surfaces. These surfaces may be distinguished using a label to describe an associated surface orientation, location, function, or some other characteristic. For example a structure may comprise one or more of an anterior surface, a posterior surface, an interior surface and an exterior surface. In another example, a seal-forming structure may comprise a face-contacting (e.g. outer) surface, and a separate non-face-contacting (e.g. underside or inner) surface. In another example, a structure may comprise a first surface and a second surface.

To facilitate describing the shape of the three-dimensional structures and the surfaces, we first consider a cross-section through a surface of the structure at a point, p. See FIG. 3B to FIG. 3F, which illustrate examples of cross-sections at point p on a surface, and the resulting plane curves. FIGS. 3B to 3F also illustrate an outward normal vector at p. The outward normal vector at p points away from the surface. In some examples we describe the surface from the point of view of an imaginary small person standing upright on the surface.

### 4.8.5.1 Curvature in One Dimension

The curvature of a plane curve at p may be described as having a sign (e.g. positive, negative) and a magnitude (e.g. 1/radius of a circle that just touches the curve at p).

Positive curvature: If the curve at p turns towards the outward normal, the curvature at that point will be taken to be positive (if the imaginary small person leaves the point p they must walk uphill). See FIG. 3B (relatively large positive curvature compared to FIG. 3C) and FIG. 3C (relatively small positive curvature compared to FIG. 3B). Such curves are often referred to as concave.

Zero curvature: If the curve at p is a straight line, the curvature will be taken to be zero (if the imaginary small person leaves the point p, they can walk on a level, neither up nor down). See FIG. 3D.

Negative curvature: If the curve at p turns away from the outward normal, the curvature in that direction at that point will be taken to be negative (if the imaginary small person leaves the point p they must walk downhill). See FIG. 3E (relatively small negative curvature compared to FIG. 3F) and FIG. 3F (relatively large negative curvature compared to FIG. 3E). Such curves are often referred to as convex.

### 4.8.5.2 Curvature of Two Dimensional Surfaces

A description of the shape at a given point on a two-dimensional surface in accordance with the present technology may include multiple normal cross-sections. The multiple cross-sections may cut the surface in a plane that includes the outward normal (a “normal plane”), and each cross-section may be taken in a different direction. Each cross-section results in a plane curve with a corresponding curvature. The different curvatures at that point may have the same sign, or a different sign. Each of the curvatures at that point has a magnitude, e.g. relatively small. The plane curves in FIGS. 3B to 3F could be examples of such multiple cross-sections at a particular point.

Principal curvatures and directions: The directions of the normal planes where the curvature of the curve takes its maximum and minimum values are called the principal directions. In the examples of FIG. 3B to FIG. 3F, the maximum curvature occurs in FIG. 3B, and the minimum occurs in FIG. 3F, hence FIG. 3B and FIG. 3F are cross sections in the principal directions. The principal curvatures at p are the curvatures in the principal directions.

Region of a surface: A connected set of points on a surface. The set of points in a region may have similar characteristics, e.g. curvatures or signs.

Edge of a surface: A boundary or limit of a surface or region.

### 4.9 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

### 4.10 Reference Signs List

1000 Patient
1100 Bed partner
3000 Patient interface
3100 Seal-forming structure
3150 Cushion module
3200 Plenum chamber
3210 Frame portion
3300 Positioning and stabilising structure
3350 Headgear structure
3351 Sleeve
3352 First layer
3354 Thermoformable layer
3354a Thermo-fusible yarn
3354b Support yarn
3356 Cavity
3359 Patient contacting portion
3360 Mandrel
3370 Rigidiser component
3380 Outer surface
3382 Inner surface
3350A Headgear structure
3351A Sleeve
3352A First layer
3353A First half
3353B Second half
3355 Second layer
3356A Cavity
3357A Lateral end regions
3358A Core
3360A Mould
3400 Vent
3600 Connection port
3700 Forehead support
3800 Unsealed patient interface
3810a Nasal prong
3810b Nasal prong
3820a Air supply lumen
3820b Air supply lumen
4000 RPT device
5000 Humidifier

1-33. (canceled)

34. A positioning and stabilising structure configured to hold a seal-forming structure of a patient interface in a sealing position on a patient's face, the positioning and stabilising structure comprising:
a headgear structure configured to extend, in use, over a portion of the patient's face and/or head, the headgear structure comprising:
a sleeve comprising an outer surface and an inner surface, wherein the outer surface of the sleeve forms an outer surface of the headgear structure which contacts the patient's face and/or head in use; and
a thermoformable layer, wherein the thermoformable layer is thermoformed to the inner surface of the sleeve to provide a pre-defined shape to the sleeve and form an inner surface of the headgear structure which surrounds a cavity extending through at least a portion of the length of the headgear structure, and wherein the outer surface of the sleeve is circumferentially continuous.

35. The positioning and stabilising structure of claim 34, wherein a) the outer surface of the sleeve is configured to be smooth, seamless and/or joint free; b) the inner surface of the sleeve is continuous.

36. The positioning and stabilising structure of claim 35, wherein the inner surface of the sleeve is configured to be smooth, seamless and/or joint free.

37. The positioning and stabilising structure of claim 34, wherein the sleeve is a continuous structure, when viewed in cross-section from a side in a direction substantially parallel to a longitudinal axis of the headgear structure.

38. The positioning and stabilising structure of claim 34, wherein the thermoformable layer comprises a thermoformable material.

39. The positioning and stabilising structure of claim 38, wherein the thermoformable material comprises a thermo-fusible yarn.

40. The positioning and stabilising structure of claim 34, wherein the thermoformable layer a) is positioned on the inner surface of the sleeve such that the thermoformable layer covers the entire inner surface of the sleeve; or b) is a continuous structure, when viewed in cross-section from a side in a direction substantially parallel to a longitudinal axis of the headgear structure; or c) has a lower melting point than a layer of the sleeve which provides the inner surface of the sleeve.

41. The positioning and stabilising structure of claim 34, wherein the headgear structure comprises a patient facing side and a non-patient facing side, wherein at least one of the patient facing side and the non-patient facing side is substantially convex shaped.

42. The positioning and stabilising structure of claim 34, wherein the thermoformable layer, when thermoformed to the inner surface of the sleeve, configures the headgear structure to be substantially resilient or to act resiliently such that the headgear structure returns to the predefined shape when flexed, bent, and/or compressed from the predefined shape.

43. The positioning and stabilising structure of claim 34, wherein the headgear structure is configured to stretch in a first direction and restrict stretching in a second direction, wherein the first direction is different to the second direction.

44. The positioning and stabilising structure of claim 43, wherein the sleeve is configured to stretch in directions which extend along the length and width of the headgear structure, and wherein the thermoformable layer is configured to stretch in a direction or directions which extend along the length of the headgear structure and restrict stretching in a direction or directions which extend along the width of the headgear structure.

45. The positioning and stabilising structure of claim 44, wherein the thermoformable material is positioned on the thermoformable layer to extend along at least a portion of the width of the thermoformable layer to restrict stretching along the width of the headgear structure.

46. The positioning and stabilising structure of claim 34, wherein the headgear structure further comprises a rigidiser component.

47. The positioning and stabilising structure of claim 46, wherein the rigidiser component is positioned inside the cavity.

48. The positioning and stabilising structure of claim 46, wherein the rigidiser component and the headgear structure are: a) permanently attached or formed together; or b) removably attached to each other.

49. The positioning and stabilising structure of claim 34, wherein a first region of the headgear structure is configured to be harder, stiffer or more rigid than a second region of the headgear structure.

50. The positioning and stabilising structure of claim 49, wherein a section of the thermoformable layer located in the first region of the headgear structure comprises a greater amount of a thermoformable material with respect to a section of the thermoformable layer located in the second region of the headgear structure.

51. A patient interface, wherein the patient interface comprises:
a plenum chamber pressurisable to a therapeutic pressure of at least 6 cmH2O above ambient air pressure, wherein the plenum chamber comprises an inlet configured to receive a flow of air at the therapeutic pressure for breathing by a patient;
a seal-forming structure configured to form a seal with a region of the patient's face surrounding an entrance to the patient's airways, wherein the seal-forming structure is configured to maintain said therapeutic pressure in the plenum chamber throughout the patient's respiratory cycle in use; and
a positioning and stabilising structure according to claim 34.
